(19) United States
(12) Patent Application Publication
(10) Pub. No.: US 2014/0004192 A1
(43) Pub. Date: Jan. 2, 2014

(54) PHARMACEUTICAL FORMULATIONS OF INDIBULIN AND USES THEREOF

(71) Applicant: Ziopharm Oncology, Inc., Boston, MA (US)

(72) Inventors: John C. Amedio, JR., Franklin, MA (US); Barbara Wallner, Cohasset, MA (US); Marsha G. Marande, Belmont, MA (US)

(21) Appl. No.: 13/796,628

(22) Filed: Mar. 12, 2013

Related U.S. Application Data
(60) Provisional application No. 61/623,964, filed on Apr. 13, 2012.
(60) Provisional application No. 61/704,882, filed on Sep. 24, 2012.

(51) Int. Cl. A61K 9/16; A61K 45/06; A61K 31/4439

(52) U.S. Cl. CPC A61K 9/1652; A61K 31/4439; A61K 45/06
USPC 424/489; 514/339; 514/266.4; 514/274; 514/49; 514/283; 424/649; 514/171; 514/34; 514/27; 514/137; 514/110

(57) ABSTRACT

The present invention relates to pharmaceutical formulations that increase the solubility and bioavailability of indibulin, such as a spray-dried solid dispersion of indibulin with at least one matrix polymer. The invention further provides dosage formulations comprising the dispersion and processes for making the dispersion. The present invention also discloses a method of treating immune system based disorders, hyper-proliferative disorders, angiogenesis, malignancies, and neoplasms with the indibulin formulations disclosed herein.

### CROSS REFERENCE TO RELATED APPLICATIONS

The present application claims priority to U.S. Application No. 61/623,964 filed on Apr. 13, 2012 and U.S. Application No. 61/704,882 filed on Sep. 24, 2012, the entire contents of these applications being incorporated herein by reference.

### BACKGROUND OF THE INVENTION

Indibulin is a synthetic small-molecule tubulin inhibitor with significant antitumor activity in vitro and in vivo. It inhibits polymerization of microtubules in tumor cells, as well as in a cell-free system. The binding site of indibulin does not appear to overlap with the tubulin-binding sites of the well-characterized microtubule-destabilizing agents taxol, vincristine and colchicine. Furthermore, the molecule selectively blocks cell cycle progression at metaphase.

In vitro, indibulin exerts significant antitumor activity against a variety of malignancies (e.g., prostate, brain, breast, pancreas, and colon). Indibulin displays high in vivo anti-neoplastic efficacy in animals. Based on its mechanism of action, it is expected to target all types of solid tumors. It is also expected to exhibit anti-asthmatic, anti-allergic, immuno-suppressant and immune-modulating actions.

Indibulin has previously been shown to have limited solubility in aqueous environments and low bioavailability when administered orally. For example, it is practically insoluble in water, methanol, ethanol or 2-propanol. A number of formulation strategies have attempted to improve indibulin oral bioavailability, with limited success. Therefore, a need exists for a new pharmaceutical formulation of indibulin which exhibits improved bioavailability of indibulin.

### SUMMARY OF THE INVENTION

The present invention relates to a spray-dried solid dispersion, or spray-dried dispersion (SDD), comprising indibulin and at least one matrix polymer. The SDD of indibulin provides enhanced solubility of indibulin in aqueous solutions, as compared to crystalline indibulin. In some embodiments, the SDD of indibulin further provides enhanced solubility of indibulin in aqueous solutions, as compared to dry blend formulations of indibulin, such as those as recited in WO2011/028743. Exemplary such formulations comprise bioavailability enhancer excipients, such as Gelucire. One dry blend formulation from WO2011/028743 (Formulation B) that may be used for comparison purposes contains by weight: 36.4% indibulin; 10% Gelucire 50/13; 5% Polysorbate 80; 45.6% Microcrystalline Cellulose; 1% Croscarmellose Sodium (added prior to granulation); 1% Croscarmellose Sodium (added after granulation was complete), 0.5% Colloidal Silicon Dioxide, and 0.5% Sodium Stearyl Fumarate.

Furthermore, in pharmacokinetic studies in rats and cancer patients, such as those provided herein, oral administration of the SDD of indibulin provides unexpectedly enhanced plasma concentration of indibulin, indicating a much improved bioavailability of the drug, as compared to orally administering an equivalent quantity of undispersed crystalline indibulin and even compared to predictions of bioavailability for spray-dried indibulin dispersion formulations. The significant enhancement in oral bioavailability of the drug reported herein was surprising and unexpected based on data and information available for indibulin.

The present invention also provides a process for making a SDD comprising indibulin, where the process includes forming a solution comprising indibulin, at least one matrix polymer, water and a water-miscible solvent in which both indibulin and at least one matrix polymer are soluble; and spray-drying the solution.

Another aspect of the present invention provides an oral dosage formulation comprising the indibulin SDD as disclosed herein. For example, the present invention provides a tablet formulation comprising the indibulin SDD.

In another aspect, the present invention provides a treatment regimen or a method for treating asthmatic, allergic, autoimmune-based conditions as well as hyper-proliferative conditions, such as cancer, and/or angiogenesis in a subject. Such methods include administering to a subject in need thereof the indibulin SDD or an oral dosage formulation comprising the dispersion. In certain embodiments, the treatment regimen or method further comprises conjointly administering to the subject one or more other therapeutic agents. In some embodiments, the combination shows efficacy that is greater than the efficacy of either agent administered alone.

In a further aspect, the present invention provides a kit comprising the indibulin SDD (e.g., as an oral dosage formulation), and a second formulation comprising at least one other therapeutic agent.

### DETAILED DESCRIPTION OF THE FIGURES

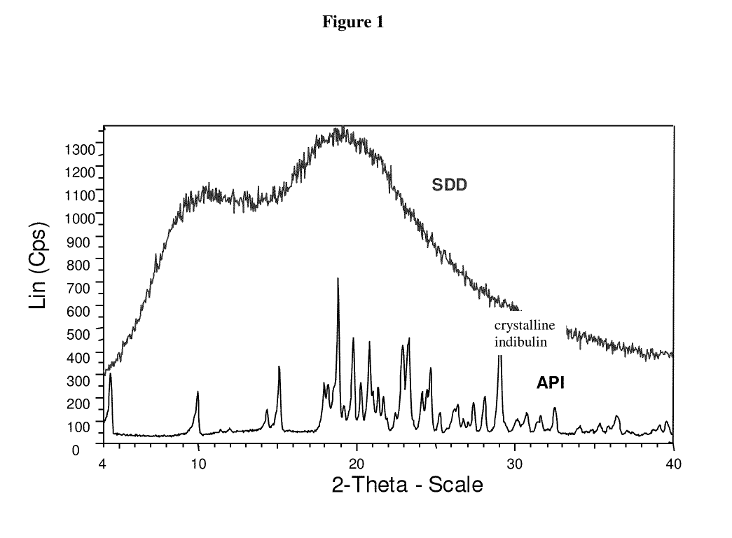

FIG. 1 shows powder X-ray diffraction patterns of crystalline indibulin and 10% active indibulin (10% A) hydroxypropyl methylcellulose acetate succinate high grade (HPMCAS-H) spray-dried dispersion (SDD).

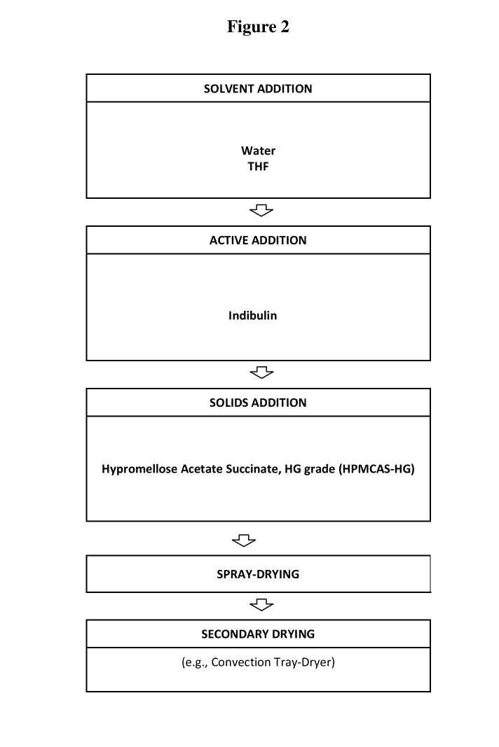

FIG. 2 is a flow chart providing an overview of the process used to manufacture the spray-dried dispersion (SDD) of indibulin according to one of the embodiments disclosed herein.

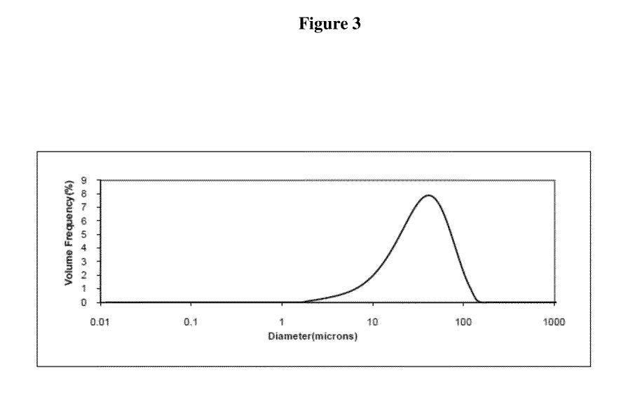

FIG. 3 shows the particle size distribution of the 10% active indibulin (10% A) hydroxypropyl methylcellulose acetate succinate high grade (HPMCAS-H) SDD: (A) summary of the volume frequency % at different particle diameter (micron). D(0.1) means that 10% of the particles in the sample have a diameter of 10 micron; D(0.5) means that 50% of the particles in the sample have a diameter of 32 micron; D(0.9) means that 90% of the particles in the sample have a diameter of 70 micron; D(3,2), which corresponds to the surface mean, is 20 micron; and D(4,3), which corresponds to the volume mean, is 37 micron; and (B) in which the particle distribution curve of volume frequency % is plotted against diameter (microns) of the indibulin SDD particles.

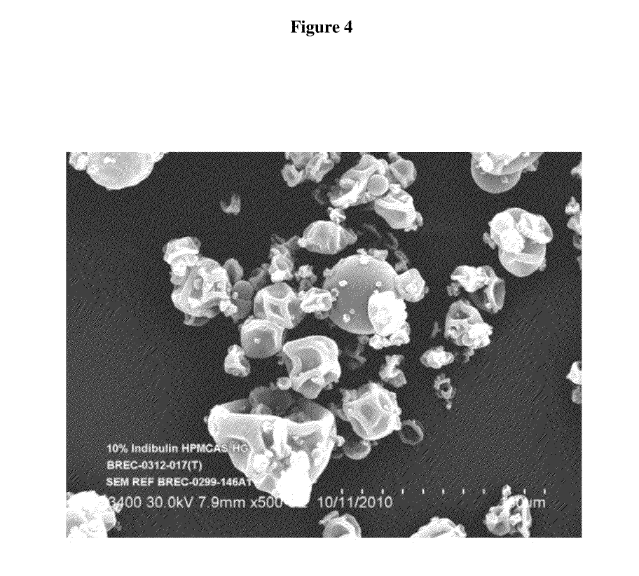

FIG. 4 shows a Scanning Electron Microscope (SEM) image of the particles of the 10% A indibulin HPMCAS SDD at 500× magnification.

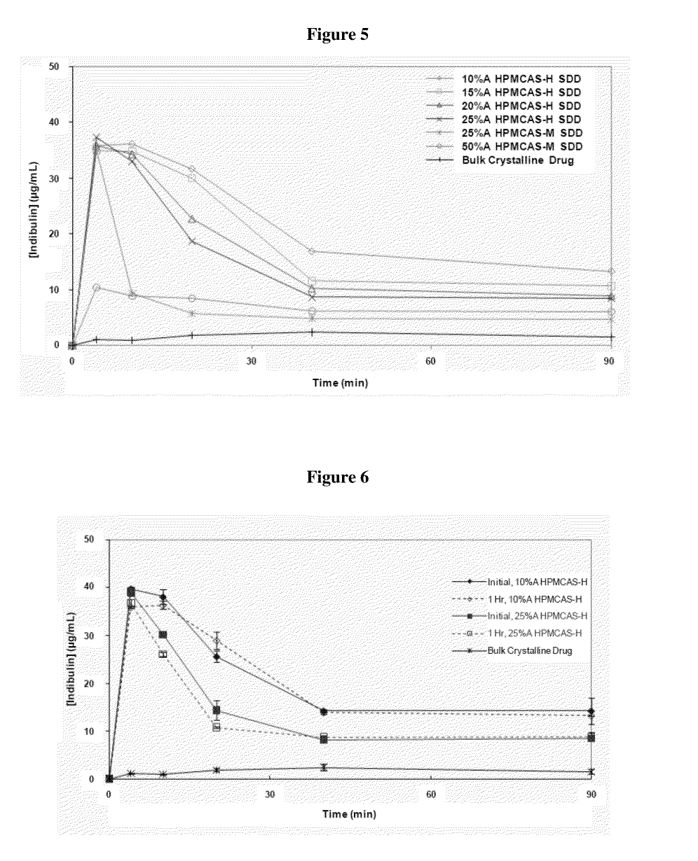

FIG. 5 shows the results of a microcentrifuge dissolution test for indibulin SDDs and crystalline indibulin, dosed at 200 μg active drug per mL (μg A/mL) into model fasted duodenal solution (MFDS), pH 6.5 in which concentration of indibulin (μg/mL) in MFDS is plotted against time (min) for the first 90 minutes following the introduction into MFDS.

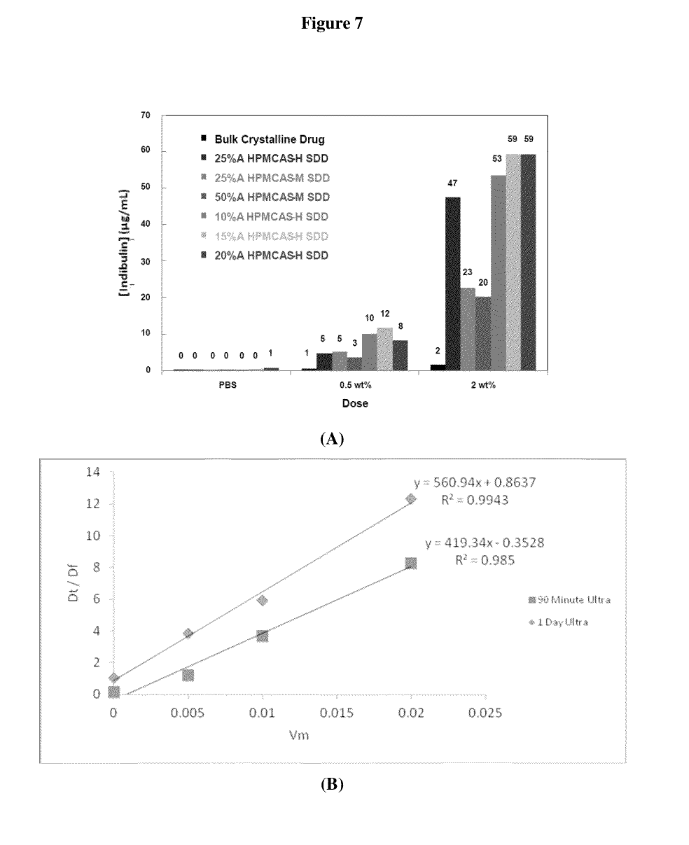

FIG. 6 shows the suspension stability of indibulin SDDs using a microcentrifuge dissolution test. In this study, indibulin SDDs and crystalline indibulin suspensions (each prepared as a 20 mgA/mL suspension in an aqueous 0.5 wt % Methocel A vehicle) are dosed at 200 μg A/mL into MFDS, pH 6.5, both immediately and 1 hour after suspension constitution, followed by a microcentrifuge dissolution test in which the concentration of indibulin (μg/mL) in MFDS is plotted against time (min) for the first 90 minutes following the introduction into MFDS.

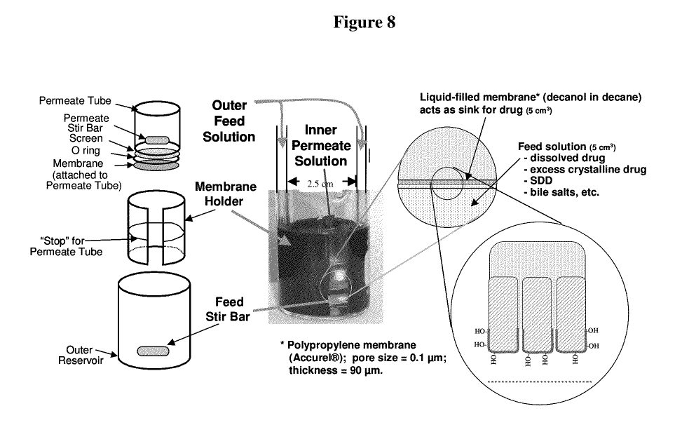

FIG. 7(A) shows the results of an ultracentrifuge dissolution test for indibulin HPMCAS SDDs and indibulin crystalline drug, dosed at 200 μgA/mL into phosphate-buffered saline (PBS) (pH 6.5), 0.5% sodium salt hydrate/1-palmitoyl-2-oleoyl-sn-glycero-3-phosphocholine (NaTC/POPC) in PBS (pH 6.5), or 2% NaTC/POPC in PBS (pH 6.5), in which test formulations are mixed for 90 minutes and centrifuged at 80,000 rpm for 20 minutes. FIG. 7(B) shows the partition coefficient result of bulk crystalline indibulin using an ultracentrifuge dissolution test, where the micelle partition coefficient (Kₚ) of the bulk crystalline indibulin is the slope of the Dₜ/D_{f} vs. Vₘ, where: Dᵣ=Average drug concentration (mg/mL); Vₘ=Wt % micelles in solution; and D_{f}=Drug concentration in aqueous solution (mg/mL).

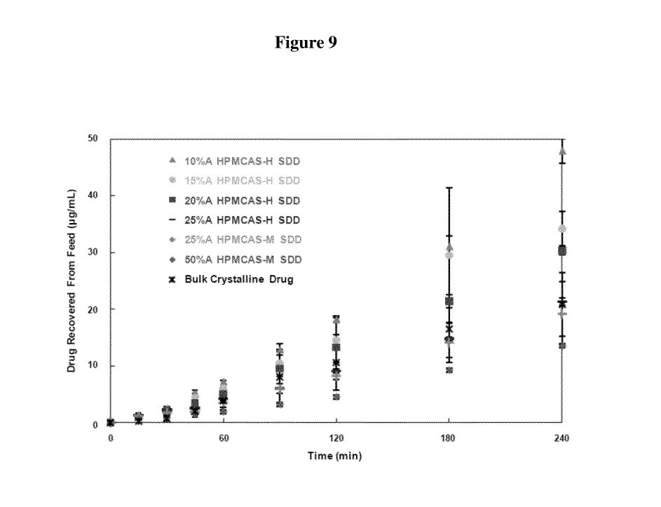

FIG. 8 shows an exemplary experimental set-up used in a membrane permeation test.

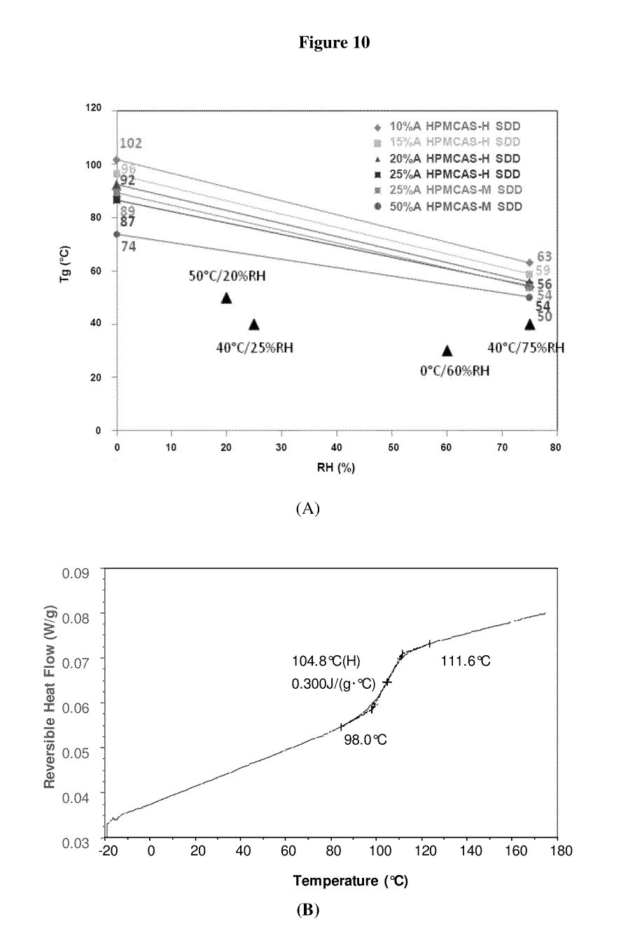

FIG. 9 shows the results of a membrane permeation test for indibulin SDDs and crystalline indibulin, dosed at 200 μgA/mL, in which the test feed is 0.5% NaTC/POPC in PBS, pH 6.5, and the sink medium is 80% decanol in decane in which the concentration of indibulin recovered from the test feed (μg/mL) is plotted against time (min).

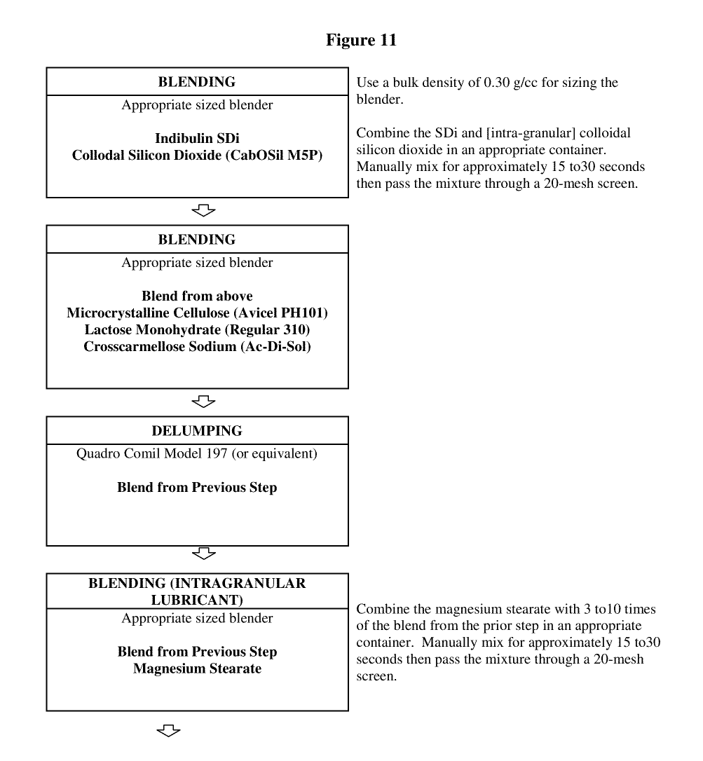

FIG. 10(A) shows the glass transition temperature (T_{g}) of indibulin SDDs as a function of humidity (RH); FIG. 10(B) shows a representative modulated differential scanning calorimetry (MDSC) Thermogram of 10% A HPMCAS-H indibulin SDD.

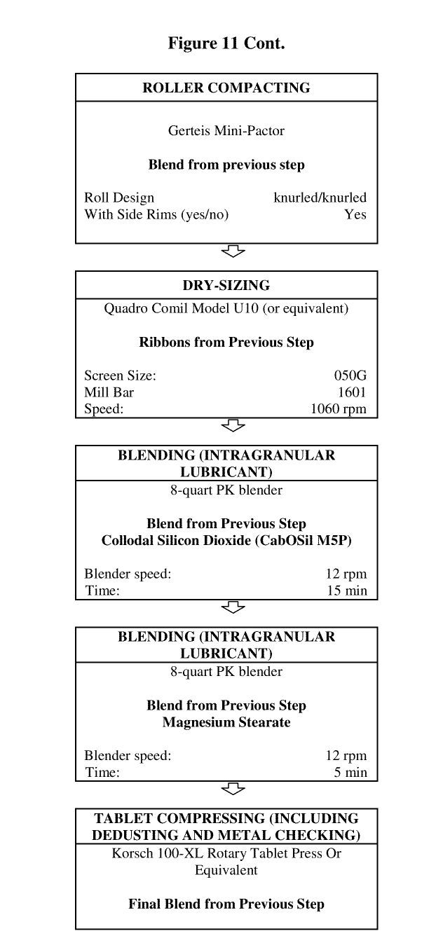

FIG. 11 is a flow chart providing an overview of the process used to manufacture the 25 mg active indibulin (25 mgA) spray-dried intermediate (SDi) tablets using 10% A HPMCAS-H indibulin SDD.

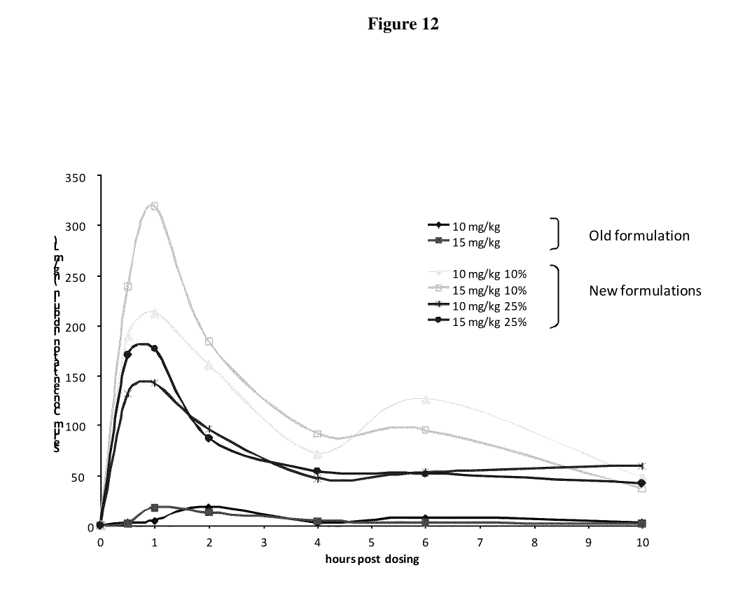

FIG. 12 shows the result of a pharmacokinetic study (first study) in rats by orally administering 10% A and 25% A indibulin HPMCAS-H SDDs, as compared to orally administering crystalline indibulin (“old formulation”).

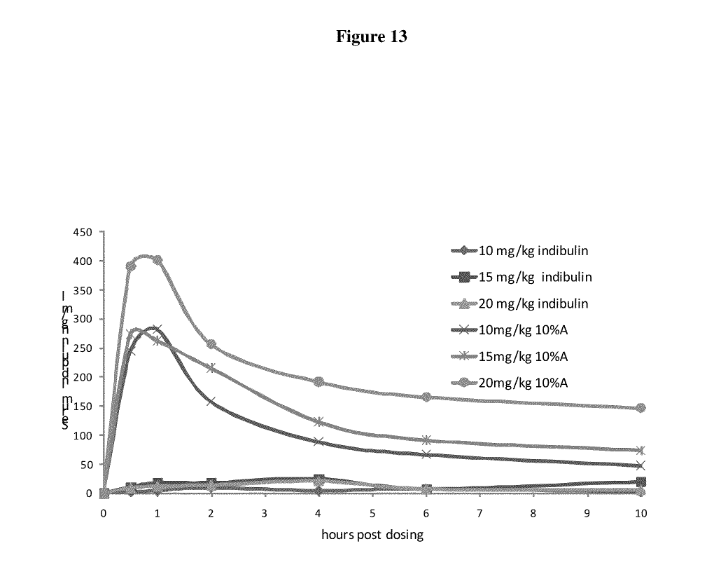

FIG. 13 shows the dose response of the plasma concentration of indibulin in rats after orally administering 10% A indibulin HPMCA-H SDD (second study), as compared to orally administering crystalline indibulin.

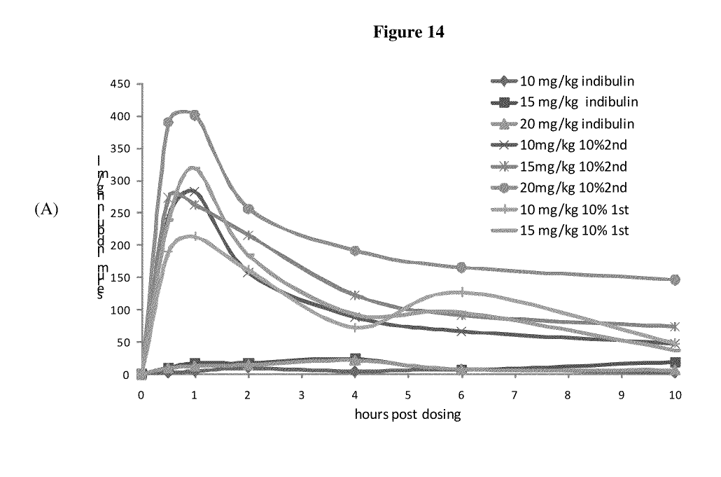

FIG. 14 shows indibulin plasma concentrations following oral administration of 10% A indibulin HPMCA-H SDD and crystalline indibulin at doses as indicated in a pharmacokinetic study (first study) and dose response study (second study) in which plasma indibulin concentration (ng/ml) is plotted against hours post dosing.

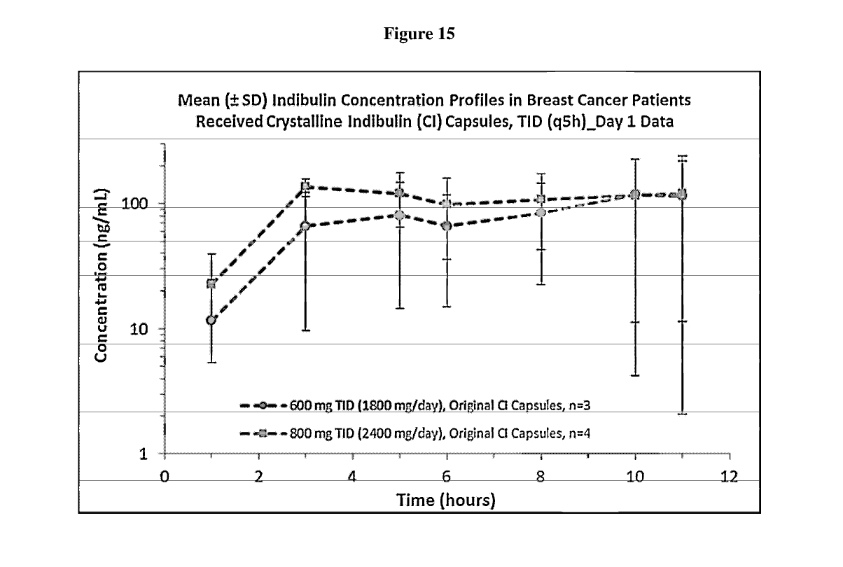

FIG. 15 shows indibulin plasma concentrations following oral administration of crystalline indibulin capsules in breast cancer patients under a fasted state. Data is shown for 600 mg, three times a day (1800 mg/day) and for 800 mg, three times a day (2400 mg/day).

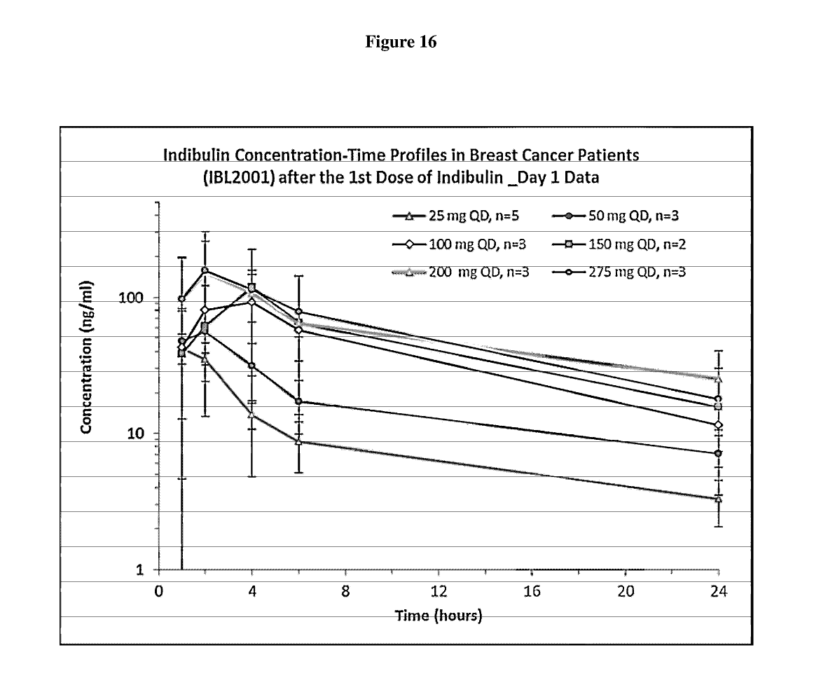

FIG. 16 shows indibulin plasma concentrations following oral administration of SDD indibulin tablets in breast cancer patients under fasted state. Data is shown for once daily doses of 25-275 mg.

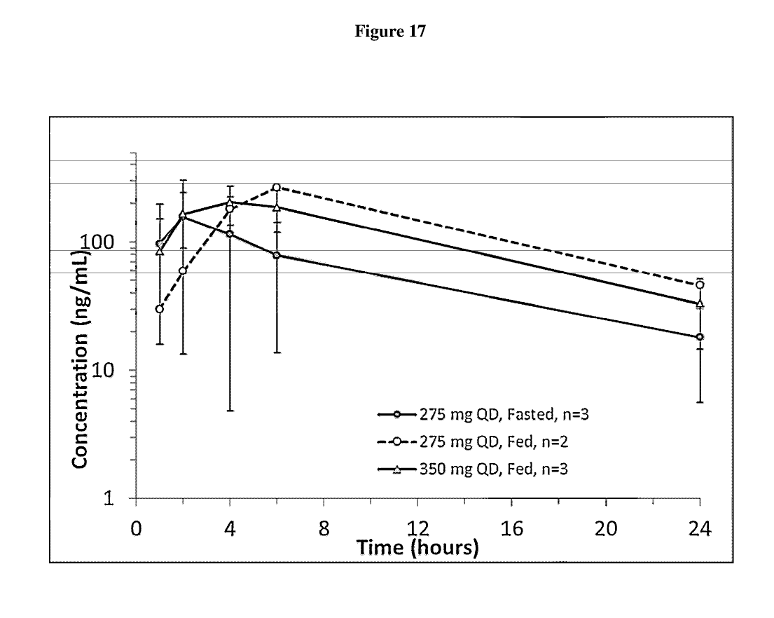

FIG. 17 shows the mean indibulin plasma concentrations following oral administration of SDD indibulin tablets in breast cancer patients under a fed state. Data is shown for once daily doses of 275 and 350 mg. Also shown is the mean plasma concentration for the 275 mg dose administered under fasted conditions.

### DETAILED DESCRIPTION OF THE INVENTION

### (a) Spray-Dried Solid Dispersions

The present invention provides a spray-dried solid dispersion (SDD) comprising indibulin and at least one matrix polymer, herein referred to as indibulin SDD, SDD indibulin or SDD of indibulin.

“Solid dispersion” as used herein refers to a solid material, in which a drug is dispersed in the solid matrix polymer. Such solid dispersions are also referred to in the art as “molecular dispersions” or “solid solutions” of the drug in the polymer.

“Spray-dried solid dispersion” or “spray-dried dispersion” (SDD), as used herein means a solid dispersion produced using spray-drying technology. The term “spray-drying” is used conventionally and refers to processes involving breaking up liquid mixtures into small droplets (atomization) and rapidly removing solvent from the mixture in a container (spray-drying apparatus), in which there is a strong driving force for evaporation of solvent from the droplets. Spray-drying processes and spray-drying equipment or apparatus are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution. Spray-drying processes and apparatus suitable for use in the present invention include those disclosed in U.S. Pat. Nos. 7,780,988 and 7,887,840, the relevant disclosures of which are incorporated herein by reference.

“Matrix polymers”, also referred to in the field as “concentration-enhancing polymers” or “dispersion polymers”, suitable for use in the present invention are discussed in detail in the U.S. Pat. Nos. 7,780,988 and 7,887,840, the relevant disclosures of which are incorporated herein by reference.

In some embodiments of the present invention, the matrix polymer used in the spray-dried dispersion is selected from hydroxypropyl methyl cellulose acetate succinate (HPMCAS), such as L, M and H grades (either as granular LG, MG and HG, or as fine powder LF, MF and HF), available from Shin-Etsu; cellulose acetate phthalate (CAP), such as the HF and CE grades available from Eastman Chemical; hydroxypropyl methyl cellulose phthalate (HPMCP), such as the NF grade available from Eastman Chemical, cellulose acetate trimellitate (CAT), available from Eastman Chemical; and hydroxypropyl methyl cellulose such as the E3 PremLV grade available from Dow. In a particular embodiment, the matrix polymer is HPMCAS, either M grade (HPMCAS-M) or H grade (HPMCAS-H). In certain embodiments of the present invention, the matrix polymer used in the spray-dried dispersion is HG (high grade granular) hydroxypropyl methyl cellulose acetate succinate (HPMCAS).

The amount of indibulin relative to the amount of matrix polymer present in the spray-dried dispersions of the present invention may vary from an indibulin-to-polymer weight ratio of about 0.01 to about 5. In some embodiments of the spray-dried dispersion, indibulin is present in the dispersion in an amount in the range of about 5% to about 60% by weight. In some embodiments, indibulin is present in an amount in the range of from about 5% to about 30%, about 5% to about 15%, about 8% to about 12% by weight, or another range within the values provided herein.

In some embodiments, the spray-dried solid dispersion of the present invention is amorphous, such as an amorphous powder.

As used herein, “amorphous” means that the solid material is in a non-crystalline state. The term “crystalline” refers to solid material in which atoms or molecules are arranged in a definite pattern that is repeated regularly in three dimensions. The term “non-crystalline” refers to solid material that is not crystalline, and therefore does not have long-range three-dimensional translational order. As used herein, material in a non-crystalline state is referred to as being in an amorphous state. The term “amorphous” is intended to include not only material which has essentially no order, but also material which may have some small degree of order, the order is in less than three dimensions and/or is only very short distances. Partially crystalline materials, liquid crystals, and disordered crystals are included as well. Amorphous or crystalline material may be characterized by techniques known in the art such as powder x-ray diffraction (PXRD), crystallography, solid state NMR, or thermal techniques such as differential scanning calorimetry (DSC).

“Crystalline indibulin”, “bulk crystalline indibulin” or “undispersed crystalline indibulin”, as used herein refers to indibulin in a crystalline powder or micronized form that has a powder X-ray diffraction pattern as shown in FIG. 1(A), where the powder X-ray diffraction pattern was determined using the conditions as specified in Table 1.

In some embodiments, the spray-dried solid dispersion as disclosed herein comprises whole and collapsed spherical particles, as observed, for example, using a Scanning Electron Microscope (SEM).

The spray-dried solid dispersion of the present invention can be a plurality of particles having an average diameter of less than 100 microns. “Average particle diameter” as used herein means volume based particle size, which equals the diameter of the sphere that has same volume as a given particle. The particle size distribution (PSD) of the dispersion disclosed herein can be determined using routine methods known in the art. Suitable methods include, for example, light scattering analysis using suitable Particle Size Analyzers, such as an LA-910 Particle Size Analyzer (Horiba Co. of Irvine, Calif.).

The present invention also provides a spray-dried solid dispersion having a bulk specific volume in the range of about 1.0 to about 10, about 2.5 to about 8.0, about 3.5 to about 7.0, or even about 4.0 to about 6.0 ml/g, or another range within the values provided herein. “Bulk specific volume” as used herein is the initial volume of the dispersion in a measuring apparatus (or container) divided by the weight, which may be expressed in ml/g (cc/g or cm³/g) dispersion.

In some embodiments, the spray-dried solid dispersion has a tapped specific volume in the range of about 0.5 to about 6.0, about 1.5 to about 4.5, or about 2.5 to about 3.5 ml/g, or another range within the values provided herein. “Tapped specific volume” means the volume of the dispersion, measured after mechanically tapping a measuring apparatus (or container) containing the dispersion, divided by the weight, which may be expressed in ml/g (cc/g or cm³/g) dispersion. Bulk and tapped specific volume of solid dispersions or powders can be measured using routine methods in the art. Suitable methods and measuring apparatus (or containers) include, for example, those disclosed in U.S. Pat. Nos. 7,780,988 and 7,887,840, the relevant disclosures of which are incorporated herein by reference.

In some embodiments of the present invention, the spray-dried dispersion has a single glass transition temperature (T_{g}) in the range of about 40 to about 140, about 50 to about 125, about 65 to about 115, or about 70 to about 110° C., or another range within the values provided herein. “Glass transition temperature (T_{g})” as used herein is the characteristic temperature where a glassy material, upon gradual heating, undergoes a relatively rapid (e.g., in about 10 to about 100 seconds) physical change from a glassy state (a hard and relatively brittle state) to a rubbery state (a rubber-like state). In some embodiments of the present invention, the spray-dried solid dispersion has a single T_{g}. A single T_{g} typically indicates that the dispersion is substantially homogeneous. This contrasts with a simple physical mixture of pure amorphous drug particles and pure amorphous polymer particles, which generally display two distinct T_{g}s, one being that of the drug and one that of the polymer. The T_{g} of an amorphous material such as a polymer, drug or dispersion can be measured by several techniques, including by a Dynamic Mechanical Analyzer (DMA), by a dilatometer, by a dielectric analyzer or by DSC. The exact values measured by each technique can vary somewhat but usually fall within about 10 to about 30° C. of each other. Regardless of the technique used, when an amorphous dispersion exhibits a single T_{g}, this typically indicates that the dispersion is substantially homogenous. Dispersions of the present invention that are substantially homogeneous generally are more physically stable and have improved concentration-enhancing properties and in turn, improved bioavailability relative to non-homogeneous dispersions.

The spray-dried dispersions of the present invention may be evaluated by in vitro dissolution tests or an in vivo relative bioavailability test. Suitable in vitro and in vivo tests for use in the present invention are discussed in the examples herein and in U.S. Pat. Nos. 7,780,988 and 7,887,840, the relevant disclosures of which are incorporated herein by reference.

In some embodiments, the spray-dried dispersions as disclosed herein provide enhanced concentrations of indibulin, relative to a control composition comprising an equivalent quantity of the undispersed crystalline indibulin, in an in vitro environment of a test solution. As used herein, the “concentration of drug” in solution refers to indibulin that may be dissolved in the form of solvated monomeric molecules (or “free drug”), or any other drug-containing submicron structure, assembly, aggregate, colloid, or micelle. Suitable test solutions for the present invention include phosphate buffered saline (PBS) or a Model Fasted Duodenal solution (MFDS). An exemplary PBS solution is an aqueous solution comprising 20 mM sodium phosphate, 47 mM potassium phosphate, 87 mM NaCl and 0.2 mM KCl, adjusted to pH 6.5. An exemplary MFDS is the same PBS solution where 7.3 mM sodium taurocholic acid and 1.4 mM of 1-palmitoyl-2-oleyl-sn-glycero-3-phosphocholine are added.

In some embodiments, the spray-dried solid dispersion as disclosed herein can be dissolution-tested by adding it to a PBS solution or an MFDS and agitating to promote dissolution. For example, the dissolution of the spray-dried solid dispersion of the present invention may be tested in a microcentrifuge test. Some embodiments of the spray-dried solid dispersion provides a maximum concentration of indibulin that is higher by a factor of at least about 5, about 8, about 10, about 12, about 15, about 20 or about 25 relative to a control composition comprising an equivalent quantity of crystalline indibulin (e.g., 200 μg indibulin per mL), in an in vitro microcentrifuge dissolution assay using a phosphate buffer solution or an MFDS of pH 6.5, at 37±0.5° C., and at centrifuge speed 50±2 rpm. In an exemplary embodiment, the control composition contains by weight: about 36.4% indibulin; about 10% Gelucire 50/13; 5% about Polysorbate 80; 45.6% about Microcrystalline Cellulose; 1% about Croscarmellose Sodium (added prior to granulation); 1% about Croscarmellose Sodium (added after granulation was complete), 0.5% about Colloidal Silicon Dioxide, and 0.5% about Sodium Stearyl Fumarate.

Alternatively, in the microcentrifuge test, the spray-dried solid dispersion of the present invention provides a dissolution area under the curve (dissolution AUC) for any period of about 90 minutes between the time of introduction into the phosphate buffer solution and about 270 minutes following introduction to the solution that is at least 1.5-fold higher than that of a dissolution AUC provided by a control composition comprising an equivalent quantity of undispersed crystalline indibulin. Dissolution AUC is the integration of a plot of the drug concentration versus time over a specified time period. For purposes of determining whether a composition or method is part of this invention, the dissolution AUC is typically calculated over a time period chosen for any time period (such as, about 90 minutes) between the time of introduction into the use environment (time=0) and about 270 minutes following introduction into the use environment. Certain embodiments of the spray-dried solid dispersion provides a dissolution AUC for the first 90 minutes following the introduction into the phosphate buffer solution that is at least about 3-, about 5-, about 8- or about 10-fold that of a dissolution AUC provided by the control composition.

The pharmacokinetic profile for the spray-dried solid dispersions as disclosed herein may be investigated in a subject (e.g., mouse, rat, dog, monkey, human). Such profile provides information regarding concentration of drug in blood plasma or serum (“C”), of the subject, generally expressed as mass per unit volume, typically nanograms per milliliter. This concentration C may also be referred to as the “drug plasma concentration”, “plasma drug concentration” or “plasma concentration”. The plasma drug concentration at any time following drug administration is referenced as Cₜᵢₘₑ, as in C₁ₕ, C₉ₕ or C₂₄ₕ, etc. A maximum plasma concentration obtained following administration of a dosage form obtained directly from the experimental data without interpolation is referred to as Cₘₐₓ. The average or mean plasma concentration obtained during a period of interest is referred to as C_{avg} or Cₘₑₐₙ. “Mean, single dose, maximum plasma concentration” means the mean Cₘₐₓ obtained over several subjects or multiple administrations to the same subject with sufficient washout in between dosings to allow drug levels to subside to pre-dose levels, following a single administration of a dosage form to each subject.

In some embodiments, orally administering the dispersion of the present invention to a rat results in a plasma Cₘₐₓ of indibulin that is higher by a factor of at least about 5 relative to orally administering a control composition comprising an equivalent quantity of bulk crystalline indibulin. In certain embodiments, the spray-dried solid dispersion provides a Cₘₐₓ of indibulin that is at least about 3-, about 4-, about 5-, about 6-, about 7-, about 8-, about 10-, about 15-, about 20-, about 25-, about 30-, about 40- or even about 80-, about 100- and about 120-fold higher relative to a control composition, when orally administered in a rat. In certain embodiments, the spray-dried solid dispersion provides a Cₘₐₓ of indibulin that is about 3-10, about 3-15, about 3-20, about 3-25, about 3-30, about 3-40, about 3-80, about 3-100, about 3-120, about 5-10, about 5-15, about 5-20, about 5-25, about 5-30, about 5-35, about 5-40, about 5-50, about 5-80, about 5-100, about 5-120, about 10-15, about 10-20, about 10-25, about 10-30, about 10-35, about 10-40, about 10-50, about 10-80, about 10-100, about 10-120, about 15-20, about 15-25, about 15-30, about 15-35, about 15-40, about 15-50, about 15-80, about 15-100, or about 15-120 fold higher relative to a control composition, when orally administered in a rat. In one embodiments, the spray-dried solid dispersion provides a Cₘₐₓ of indibulin that is 5-30, 10-35, 12-33, or 15-35 fold higher relative to a control composition, when orally administered in a rat.

Similarly, in some embodiments, orally administering the dispersion of the present invention to a human results in a plasma Cₘₐₓ of indibulin that is higher by a factor of at least about 3 relative to orally administering a control composition comprising an equivalent quantity of bulk crystalline indibulin. In certain embodiments, the spray-dried solid dispersion provides a Cₘₐₓ of indibulin that is at least about 3-, about 4-, about 5-, about 6-, about 7-, about 8-, about 10-, about 15-, about 20-, about 25-, about 30-, about 40- or even about 80-, about 100- and about 120-fold higher relative to a control composition, when orally administered in a human. In certain embodiments, the spray-dried solid dispersion provides a Cₘₐₓ of indibulin that is about 3-10, about 3-15, about 3-20, about 3-25, about 3-30, about 3-40, about 3-80, about 3-100, about 3-120, about 5-10, about 5-15, about 5-20, about 5-25, about 5-30, about 5-35, about 5-40, about 5-50, about 5-80, about 5-100, about 5-120, about 7-10, about 7-15, about 7-20, about 7-25, about 7-30, about 7-35, about 7-40, about 7-50, about 7-80, about 7-100, about 7-120, about 10-15, about 10-20, about 10-25, about 10-30, about 10-35, about 10-40, about 10-50, about 10-80, about 10-100, about 10-120, about 15-20, about 15-25, about 15-30, about 15-35, about 15-40, about 15-50, about 15-80, about 15-100, or about 15-120 fold higher relative to a control composition, when orally administered in a human. In one embodiments, the spray-dried solid dispersion provides a Cₘₐₓ of indibulin that is about 5-30, about 10-35, about 12-33, or about 15-35 fold higher relative to a control composition, when orally administered in a human.

The pharmacokinetic profile for the spray-dried solid dispersions of the present invention also provides an area under the curve (or plasma AUC), which is the area as measured under a plasma drug concentration curve. The plasma AUC is sometimes specified in terms of the time interval across which the plasma drug concentration curve is being integrated, for instance AUC_{start-finish}. For example, AUC₀₋₄₈ refers to the AUC obtained from integrating the plasma concentration curve over a period of zero to 48 hours, where zero is conventionally the time of administration of the drug or dosage form comprising the drug to a subject.

In some embodiments, orally administering the dispersion of the present invention to a rat results in an area under the curve (AUC) for plasma indibulin that is higher by a factor of at least about 5 relative to orally administering a control composition comprising an equivalent quantity of bulk crystalline indibulin. In certain embodiments, the dispersion of the present invention provides an AUC for plasma indibulin that is at least about 7-, about 8-, about 9-, about 10-, about 15-, about 18-, about 20-, about 25-, about 35-, about 38-, or about 45-, or even about 80-, about 100- and about 120-fold higher relative to the control composition, when orally administered in a rat. In certain embodiments, the spray-dried solid dispersion of the present invention provides an AUC for plasma indibulin that is at least about 7-10, about 7-15, about 7-20, about 7-25, about 7-30, about 7-35, about 7-40, about 7-50, about 7-80, about 7-100, about 7-120, 8-10, about 8-15, about 8-20, about 8-25, about 8-30, about 8-35, about 8-40, about 8-50, about 8-80, about 8-100, about 8-120, about 9-10, about 9-15, about 9-20, about 9-25, about 9-30, about 9-35, about 9-40, about 9-50, about 9-80, about 9-100, about 9-120, about 10-15, about 10-20, about 10-25, about 10-30, about 10-35, about 10-40, about 10-50, about 10-80, about 10-100, about 10-120, about 15-20, about 15-25, about 15-30, about 15-35, about 15-40, about 15-50, about 15-80, about 15-100, or about 15-120 fold higher relative to the control composition, when orally administered in a rat. In certain embodiments, the dispersion of the present invention provides an AUC for plasma indibulin that is at least about 8-25, about 9-20, about 9-22, about 9-25, about 10-25 fold higher relative to the control composition, when orally administered in a rat.

Similarly, in some embodiments, orally administering the spray-dried solid dispersion of the present invention to a human results in an area under the curve (AUC) for plasma indibulin that is higher by a factor of at least about 5 relative to orally administering a control composition comprising an equivalent quantity of bulk crystalline indibulin. In certain embodiments, the dispersion of the present invention provides an AUC for plasma indibulin that is at least about 8-, about 9-, about 10-, about 15-, about 18-, about 20-, about 25-, about 35-, about 38-, or about 45-, or even about 80-, about 100- or about 120-fold higher relative to the control composition, when orally administered in a human. In certain embodiments, the dispersion of the present invention provides an AUC for plasma indibulin that is at least about 8-10, about 8-15, about 8-20, about 8-25, about 8-30, about 8-35, about 8-40, about 8-50, about 8-80, about 8-100, about 8-120, about 9-10, about 9-15, about 9-20, about 9-25, about 9-30, about 9-35, about 9-40, about 9-50, about 9-80, about 9-100, about 9-120, about 10-15, about 10-20, about 10-25, about 10-30, about 10-35, about 10-40, about 10-50, about 10-80, about 10-100, about 10-120, about 15-20, about 15-25, about 15-30, about 15-35, about 15-40, about 15-50, about 15-80, about 15-100, or about 15-120 fold higher relative to the control composition, when orally administered in a human. In certain embodiments, the dispersion of the present invention provides an AUC for plasma indibulin that is at least about 8-25, about 9-20, about 9-22, about 9-25, or about 10-25 fold higher relative to the control composition, when orally administered in a human.

In an embodiment, the SDD indibulin compositions provides plasma exposures that are about 5-10, about 5-20, about 5-25, about 5-30, about 5-35, about 5-40, about 10-20, about 10-25, about 10-30, about 10-35, about 10-40, about 15-20, about 15-25, about 15-30, about 15-35, or about 15-40 fold higher when compared to the control composition. Plasma exposure can be measured using variables such as Cₘₐₓ and/or AUC. In another embodiment, the oral bioavailability of SDD indibulin is at least about 5-30, about 10-20, about 10-25, or about 15-30, or about 15-40 fold higher when compared to the control composition.

In another embodiment, the time for maximum plasma concentration to occur (tₘₐₓ) for SDD indibulin is about 0.5-7 hours, about 1-7 hours, about 1.5-7 hours, about 2-7 hours, about 3-7 hours, about 0.5-6 hours, about 1-6 hours, about 1.5-6 hours, about 2-6 hours, about 3-6 hours, 0.5-5 hours, about 1-5 hours, about 1.5-5 hours, about 2-5 hours, about 3-5 hours, about 0.5-4 hours, about 1-4 hours, about 1.5-4 hours, about 2-4 hours, about 0.5-3 hours, about 1-3 hours, about 1.5-3 hours, or about 2-3 hours. In another embodiment, the time for maximum plasma concentration to occur (tₘₐₓ) for SDD indibulin is about 0.5 hour, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, or about 7 hours post administration.

In one embodiment, commonly observed adverse events in subjects administered with the SDD indibulin compositions include anorexia, constipation, cough, nausea, dyspnea, fatigue, vomiting, decreased appetite, and/or diarrhea.

### (b) Spray-Drying Processes

In another aspect, the invention relates to a process for making a spray-dried solid dispersion as disclosed herein, the process comprising the steps of (a) forming a solution comprising indibulin, at least one matrix polymer, water and a water-miscible solvent in which both indibulin and the at least one matrix polymer are soluble; and (b) spray-drying the solution of step (a).

“Water-miscible solvent” as used herein means solvent which is miscible with water at a solvent concentration of less than about 50 wt % of the solvent/water mixture. Such solvents are well known in the art. Examples of water miscible solvents suitable for the present invention include, without limitation, alcohols such as methanol and ethanol; ketones such as acetone and various other solvents such as acetonitrile, and tetrahydrofuran (THF), and the like.

In some embodiments, the water-miscible solvent used in the process of the present invention is THF. In certain embodiments, THF and water used in step (a) of the spray-drying process are in a ratio in the range of 65:35 to 99:1 or 90:10 to 99:1 w/w, or another range within the values provided herein.

SDD of indibulin are generally prepared as follows. Crystalline indibulin and matrix polymer are dissolved in a water-solvent solution to form a spray solution. The water and solvent are subsequently removed during spray-drying using a small-scale spray-drying apparatus such as a GEA-Niro Mobil Minor spray dryer, resulting in a homogenous dispersion in powder form. Examples of suitable small-scale spray-drying apparatus for use in the present invention may be found in U.S. Pat. No. 7,780,988 and U.S. Patent Application Publication No. 2010/0029667 (corresponding to U.S. patent application Ser. No. 12/311,543). FIG. 2 is a flow chart providing an overview of an exemplary process used to manufacture the SDD of indibulin using a GEA-Niro Mobil Minor spray dryer.

In a first step, a water-solvent mixture is formed. In one embodiment, water and THF are added. In some embodiments, water and THF are added in a stainless-steel tank equipped with a top-mounted mixer. In some embodiments, agitation is achieved using a top-mounted mixer. Indibulin is subsequently added to the mixture. In some embodiments, indibulin is added while a mixer, such as a top-mounted mixer provides adequate agitation. In some embodiments, the solution with indibulin is mixed for a specified period of time. In the embodiment of FIG. 2, solids are then added to the mixture. Again, in some embodiments, the solids are added while a mixer, such as a top-mounted mixer provides adequate agitation. The solution is mixed for a specified period of time after addition of the solids. In some embodiments, the solids include hypromellose. In certain embodiments, the hypromellose is a hypromellose salt. Exemplary hypromellose salts include, without limitation, hypromellose acetate succinate (HPMCAS) (e.g., HPMCAS-HG (high grade)). The mixture is then spray dried under appropriate conditions.

In some embodiments, the solvent used for the spray drying includes THF. In some embodiments, 95/5 THF/water is used for warmup and shutdown of the spray dryer. In some embodiments, the mixture is filtered prior to being added to the spray drier. In some embodiments, the filter is ≦100 μm, ≦150 μm, ≦200 μm, ≦250 μm, ≦300 μm, ≦350 μm, ≦400 μm, ≦450 μm, ≦500 μm. In certain embodiments, the material is processed though a secondary drying step. In some embodiments, a tray dryer is used for secondary drying. For example, in some embodiments, the dryer is a convention dryer. The secondary drying is performed for a sufficient period of time to meet product specifications. For example, in some embodiments, secondary drying occurs at 30° C., 35° C., 40° C., 45° C., or 50° C. In some embodiments, the temperature of the secondary drying is from about 35° C. to about 45° C. In some embodiments, the humidity of the secondary drying is controlled. For example, in some embodiments, the humidity is about 40% RH, about 45% RH, about 50% RH, about 55% RH, or about 60% RH. In some embodiments, the secondary drying is performed at about 40-60% RH. In certain embodiments, the drying time is at least about 2, 3, 5, 6, 7, 8, 9, or 10 hours. In certain embodiments, the drying time is about 2-15 hours, 4-12 hours, 5-10 hours or 6-8 hours.

### (c) Dosage Formulations

A “dosage form” or “dosage formulation” as used herein means a unit of administration of an active agent. Examples of dosage formulations include tablets, granules, capsules, injections, suspensions, liquids, emulsions, creams, ointments, suppositories, inhalable formulations, transdermal formulations, and the like. By “oral dosage formulation” is meant to include a unit dosage formulation for oral administration.

In some embodiments, the SDD of the present invention is combined with one or more optional excipients to formulate the dispersion into suitable dosage formulations, such as tablets, capsules, suspensions, powders for suspensions, cream, transdermal patches, depots, and the like. The dispersion can also be added to other dosage form ingredients in a manner that advantageously does not substantially alter the indibulin's activity.

Generally, excipients such as surfactants, pH modifiers, fillers, matrix materials, complexing agents, solubilizers, lubricants, glidants, and so forth may be used for customary purposes and in typical amounts without adversely affecting the properties of the compositions. See for example, Remington's Pharmaceutical Sciences (18th ed. 1990).

The addition of pH modifiers such as acids, bases, or buffers may be beneficial, retarding the dissolution of the composition (e.g., acids such as citric acid or succinic acid when the matrix polymer is anionic) or, alternatively, enhancing the rate of dissolution of the composition (e.g., bases such as sodium acetate or amines when the matrix polymer is cationic).

Conventional matrix materials, complexing agents, solubilizers, fillers, diluents, disintegrating agents (disintegrants), preservatives, suspending agents or thickeners, anti-caking agents, or binders may also be added as part of the composition itself or added by granulation via wet or mechanical or other means. These materials may comprise up to 90 wt % of the composition.

Examples of matrix materials, fillers, or diluents include, without limitation, lactose, mannitol, xylitol, microcrystalline cellulose, dibasic calcium phosphate (anhydrous and dihydrate) and starch.

Examples of disintegrants include, without limitation, sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE (available from BASF Corporation).

Examples of binders include, without limitation, methyl cellulose, microcrystalline cellulose, starch, and gums such as guar gum, and tragacanth.

Examples of lubricants include, without limitation, magnesium stearate, calcium stearate, and stearic acid.

Examples of the glidant include, without limitation, metal silicates, silicon dioxides, higher fatty acid metal salts, metal oxides, alkaline earth metal salts, and metal hydroxides. Examples of preservatives include, without limitation, sulfites (an antioxidant), benzalkonium chloride, methyl paraben, propyl paraben, benzyl alcohol and sodium benzoate.

Examples of suspending agents or thickeners, without limitation, include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, and titanium dioxide.

Examples of anti-caking agents or fillers, without limitation, include silicon oxide and lactose.

Examples of solubilizers include, without limitation, ethanol, propylene glycol or polyethylene glycol.

One other class of excipients is surfactants, optionally present from about 0 to about 10 wt %. Suitable surfactants include, without limitation, fatty acid and alkyl sulfonates; commercial surfactants such as benzalkonium chloride (HYAMINE™ 1622, available from Lonza, Inc., Fairlawn, N.J.); dioctyl sodium sulfosuccinate (DOCUSATE SODIUM, available from Mallinckrodt Spec. Chem., St. Louis, Mo.); polyoxyethylene sorbitan fatty acid esters (TWEEN™, available from ICI Americas Inc., Wilmington, Del.; LIPOSORB™ O-20, available from Lipochem Inc., Patterson N.J.; CAPMUL™ POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and diglycerides. Such materials can be employed to increase the rate of dissolution by, for example, facilitating wetting, or otherwise increase the rate of drug release from the dosage form.

Other conventional excipients, including pigments, lubricants, flavorants, humectants, solution retarding agents, absorption accelerators, wetting agents, absorbents, and other ones well-known in the art, may be employed in the compositions of this invention. For example, excipients such as pigments, lubricants, flavorants, and so forth may be used for customary purposes and in typical amounts without adversely affecting the properties of the compositions.

In one embodiment, the SDD compositions provided comprises indibulin, hypromellose acetate succinate, microcrystalline cellulose, lactose monohydrate, croscarmelose sodium, colloidal silicon dioxide, and magnesium stearate.

The spray-dried solid dispersion of the present invention may be used in a wide variety of dosage forms for administration by a wide variety of routes, including, but not limited to, oral, nasal, rectal, vaginal, transdermal, buccal, subcutaneous, intravenous, and pulmonary.

In certain embodiments, the spray-dried solid dispersion as disclosed herein is formulated as an oral dosage formulation. Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an elixir or syrup, or as pastilles (using an inert matrix, such as gelatin and glycerin, or sucrose and acacia), and the like, each containing a predetermined amount of an active ingredient. A composition may also be administered as a bolus, electuary, or paste.

In one embodiment, the oral dosage formulation of the present invention is a tablet. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. The active ingredient can also be in micro-encapsulated form, if appropriate, with one or more of the above-described excipients.

An overview of the manufacturing process for an indibulin SDD tablet is shown in FIG. 11. Indibulin SDD is mixed with one or more excipients in one or more blending steps. In some embodiments, there are two or three blending steps. In certain embodiments, a build density of 0.30 g/cc is used. In some embodiments, one or more of the blending steps is preceded by manual blending for a certain period of time (e.g., at least about 10 secs, 15 secs, 20 secs, 30 secs, or 1 minute). In certain embodiments, the preblended ingredients are passed through a screen (e.g., 10-mesh, 15-mesh, 20-mesh, 25-mesh, or 30-mesh) before being added to the blender. In exemplary embodiments, the blending steps are performed at a speed of about 10-20 rpm, or about 10-15 rpm. In some exemplary embodiments, the blending steps are performed at a speed of about 10 rpm, 11 rpm, 12 rpm, 13 rpm, 14 rpm, or 15 rpm. In nonlimiting exemplary embodiments, each blending step is performed for at least 5, 10, 15, or 20 minutes. In other embodiments, the blending step is performed for about 5-20 minutes, or 5-15 minutes. In other embodiments, the blending step is performed for about 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, or 20 minutes. As shown in FIG. 11, in certain embodiments, there is a delumping step. In the embodiment of FIG. 11, the delumping step occurs in between two of the blending step, though in other embodiments, the delumping step occurs after all blending is complete, or at another appropriate time in the process. In certain embodiments, a comill is used for delumping (e.g., affixed with a 032R screen size, a 1601 mill impellor, at about 1500 rpm). As shown in FIG. 11, the mixture undergoes roller compacting. In certain embodiments, the blend is roller compacted to a target solid fraction of 0.65 (±0.02) using a true density of 1.400 g/cm². In certain embodiments, a comill (e.g., Quadro Comil Model U10) is used for further blending. In blending the intragranular lubricant, in certain embodiments, a bulk density of 0.40 g/cc is used for sizing the blender. Blending of the intragranular lubricant is performed in one or more steps. In certain embodiments, the excipient (e.g., [extra-granular] colloidal silicon dioxide) is combined with 3 to 10 times of the blend from the prior step in an appropriate container. In certain embodiments, a further intragranular lubricant (e.g., magnesium stearate) is combined with about 3 to 10 times of the blend from the prior step in an appropriate container.

In certain embodiments, the oral dosage formulation of the present invention comprises a filler, a disintegrant, a glidant and a lubricant.

In some embodiments of the oral dosage formulation as disclosed herein, the spray-dried solid dispersion is present in an amount of from about 20 to about 80%, about 30 to about 60% or about 45 to about 55% by weight, or another range within the values provided herein. In these and other embodiments of the oral dosage formulation, indibulin can be present in an amount of from about 10 to about 150 mg per dose unit. In one embodiment, the indibulin can be present in an amount of 25 mg per dose unit. In one embodiment, the indibulin can be present in an amount of 50 mg per dose unit. In one embodiment, the indibulin can be present in an amount of 100 mg per dose unit.

The spray-dried solid dispersion and the dosage formulation as disclosed herein may be used to treat any condition that is subject to treatment by administering indibulin, including conditions discussed in U.S. Patent Application Publication Nos. 2006/0280787 and 2008/0241274, the relevant disclosures of which publications are incorporated herein by reference. For example, conditions that may be treated by the dispersion or dosage form of the present invention include hyperproliferative disorders, malignancies and neoplasms (e.g., solid tumors). Such hyperproliferative disorders, malignancies, and neoplasms include, but are not limited to, one or more of the following: drug-resistant or metastasizing carcinoma (including development and spread of metastases, tumors sensitive to angiogenesis inhibitors or tumors that are both antitumor agent-resistant and sensitive to angiogenesis inhibitors), adenoid cystic carcinoma, renal cell carcinoma, glioblastoma, cancers of the abdomen, blood, bone, breast, digestive system, liver, lung, pancreas, peritoneum, prostate, vulvar, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Other hyperproliferative disorders include, but are not limited to, angiogenesis, hypergammaglobulinemia, lymphoproliferative disorders, araproteinemias, purpura, sarcoidosis, Sezary syndrome, Waldenstrom's macroglobulinemia, Gaucher's disease, histiocytosis, and any other hyperproliferative disease, besides neoplasia, located in an organ system listed above. Further conditions that may be treated by the dispersion or dosage form of the present invention include asthma or allergies. In certain embodiments, the dispersion or dosage form as described herein also may be used for suppressing or inducing regression of an immunological response in a subject.

The present invention further provides a method of treating cancer, wherein the method further comprising conjointly administering to the subject one or more other (or additional) therapeutic agents, wherein the combination shows efficacy that is greater than the efficacy of either agent administered alone. For example, in one nonlimiting embodiment, the combination is a synergistic combination. In another nonlimiting embodiment, the combination is an additive combination.

The term “therapeutic agent” is art-recognized and refers to any chemical moiety that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human. In certain embodiments, the therapeutic agents conjointly administered with indibulin are anti-cancer agents, such as conjoint therapies disclosed in U.S. Patent Application Publication No. 2008/0241274, the relevant disclosure of which is incorporated herein by reference.

For example, additional therapeutic agents that can be used in combination with the spray-dried solid dispersion and the dosage formulation as disclosed herein include microtubule binding agents, DNA intercalators or cross-linkers, DNA synthesis inhibitors, DNA and/or RNA transcription regulators, antibodies, enzymes, enzyme inhibitors, gene regulators, and/or angiogenesis inhibitors.

Microtubule binding agent refers to an agent that interacts with tubulin to stabilize or destabilize microtubule formation thereby inhibiting cell division. Examples of microtubule binding agents that can be used in combination with the presently disclosed spray-dried solid dispersion and the dosage formulation include, but are not limited to, paclitaxel, docetaxel, vinblastine, vincristine vindesine, vinorelbine (navelbine), the epothilones, colchicine, dolastatin 15, nocodazole, podophyllotoxin, rhizoxin, cryptophycin, estramustine, indibulin and analogues or derivatives thereof.

Suitable DNA and/or RNA transcription regulators, including, but not limited to, actinomycin D, daunorubicin, doxorubicin and derivatives and analogs thereof also are suitable for use in combination with the presently disclosed spray-dried solid dispersion and the dosage formulation.

DNA intercalators and cross-linking agents useful in certain embodiments include, but are not limited to, cisplatin, carboplatin, oxaliplatin, mitomycins, such as mitomycin C, bleomycin, chlorambucil, cyclophosphamide, palifosfamide, ifosfamide, and derivatives and analogs thereof.

DNA synthesis inhibitors suitable for use as therapeutic agents include, but are not limited to, methotrexate, 5-fluoro-5′-deoxyuridine, 5-fluorouracil, gemcitabine, capecitabine and analogs thereof.

Examples of suitable enzyme inhibitors for use in combination with the presently disclosed spray-dried solid dispersion and the dosage formulation include, but are not limited to, erlotinib, camptothecin, etoposide, formestane, trichostatin and derivatives and analogs thereof.

Suitable therapeutics for use with the presently disclosed spray-dried solid dispersion and dosage formulation, which affect gene regulation include agents that result in increased or decreased expression of one or more genes, include, but are not limited to, raloxifene, 5-azacytidine, 5-aza-2′ deoxycytidine, tamoxifen, 4-hydroxytamoxifen, mifepristone and derivatives and analogs thereof.

Angiogenesis inhibitors are known in the art and examples of suitable angiogenesis inhibitors for use in the present invention include, but are not limited to, angiostatin K1-3, staurosporine, genistein, fumagillin, medroxyprogesterone, suramin, interferon-alpha, metalloproteinase inhibitors, platelet factor 4, somatostatin, thrombospondin, endostatin, thalidomide, bevacizumab, sorafenib, sunitinib, pazopanib, everolimus and derivatives and analogs thereof.

Other therapeutic agents, particularly anti-cancer or anti-tumor agents, that may or may not fall under one or more of the categories above, also are suitable for administration in combination with the presently disclosed compounds. By way of example, such agents include adriamycin, apigenin, rapamycin, zebularine, cimetidine, prednisolone and derivatives and analogs thereof.

In some embodiments, the spray-dried solid dispersion or the dosage formulation is administered in combination with one or more other therapeutic agents selected from vinca alkaloids (e.g., vinblastine, vincristine, and vinorelbine), taxanes (e.g., paclitaxel and docetaxel), epipodophyllotoxins (e.g., etoposide, teniposide), antibiotics (e.g., dactinomycin (actinomycin D), daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, ifosphamide, palifosfamide, cyclophosphamide and analogs, melphalan, chlorambucil), ethylenimines and methylmelamines (e.g., hexamethylmelamine and thiotepa), alkyl sulfonates (busulfan), nitrosoureas (e.g., carmustine (BCNU) and analogs, streptozocin), trazenes-dacarbazinine (DTIC); antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine); aromatase inhibitors (e.g., anastrozole, exemestane, and letrozole); and platinum coordination complexes (e.g., cisplatin, carboplatin), procarbazine, hydroxyurea, mitotane, aminoglutethimide; hormones (e.g., estrogen) and hormone agonists such as leutinizing hormone releasing hormone (LHRH) agonists (e.g., goserelin, leuprolide and triptorelin), and organic arsenicals (e.g., darinaparsin). Other chemotherapeutic agents may include mechlorethamine, camptothecin, ifosfamide, tamoxifen, raloxifene, gemcitabine, navelbine, or any analog or derivative variant of the foregoing.

In certain embodiments, the one or more other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, prednisolone, palifosfamide, ifosfamide, and darinasparin.

“Treating” a condition or disease refers to curing as well as ameliorating at least one symptom of the condition or disease. Treating includes administration of a composition which reduces the frequency of, or delays the onset of, symptoms of a medical condition in a subject relative to a subject which does not receive the composition. Thus, treatment of cancer includes, for example, reducing the number and/or size of detectable cancerous growths in a population of patients receiving a treatment relative to an untreated control population, and/or delaying the appearance of detectable cancerous growths in a treated population versus an untreated control population, e.g., by a statistically and/or clinically significant amount.

A “patient”, “subject”, “individual” or “host” refers to either a human or a non-human animal.

Another aspect of the invention relates to a kit comprising the spray-dried solid dispersion or the dosage formulation as disclosed herein, and a second formulation comprising at least one other therapeutic agent. The other therapeutic agent may be any of the ones discussed above. In some embodiments, the other therapeutic agent suitable for use in the kit is one or more of erlotinib, carboplatin, 5-fluorouracil, capecitabine, paclitaxel, tamoxifen, vinorelbine, cisplatin, gemcitabine, estramustine, doxorubicin, vinblastine, etoposide, prednisolone, palifosfamide, ifosfamide, or darinasparin.

In some embodiments, SDD indibulin compositions described herein are administered at total daily doses of less than about 500 mg, less than about 400 mg, less than about 300 mg, less than about 250 mg, less than about 200 mg, less than about 150 mg, or less than about 100 mg.

In certain embodiments, the SDD indibulin compositions described herein are administered at total daily doses of about 5 mg/kg to about 25 mg/kg. In some embodiments, the compositions described herein are administered at total daily doses of about 10 mg/kg to about 25 mg/kg, or about 15 mg/kg to about 25 mg/kg, or about 10 mg/kg to about 20 mg/kg, or about 10 mg/kg to about 15 mg/kg. In some embodiments, the compositions are administered at total daily doses of about 5 mg/kg, about 10 mg/kg, about 15 mg/kg, about 20 mg/kg, or about 25 mg/kg.

One of ordinary skill in the art will be able to obtain conversions of animal and human equivalent based on body surface area (BSA). For example, human BSA can be calculated using the following: BSA (m²)=SQRT([height (cm)×weight (kg)]/3600) or BSA (m²)=SQRT([height (in)×weight (lbs)]/3131). Additional general guidance can be publically found from the U.S. Food and Drug Administration, for example, on their website (www.fda.gov/downloads/Drugs/ . . . /Guidances/UCM078932.pdf).

In other embodiments, the SDD indibulin compositions described herein are administered at total daily doses of about 5 mg to about 500 mg, or about 5 mg to about 400 mg, or about 5 mg to about 300, or about 5 mg to about 200 mg, or about 5 mg to about 150 mg, or about 5 mg to about 100 mg, or about 10 mg to about 500 mg, or about 10 mg to about 400 mg, or about 10 mg to about 300, or about 10 mg to about 200 mg, or about 10 mg to about 150 mg, or about 10 mg to about 100 mg, or about 15 mg to about 500 mg, or about 15 mg to about 400 mg, or about 15 mg to about 300, or about 15 mg to about 200 mg, or about 15 mg to about 150 mg, or about 15 mg to about 100 mg, or about 20 mg to about 500 mg, or about 20 mg to about 400 mg, or about 20 mg to about 300, or about 20 mg to about 200 mg, or about 20 mg to about 150 mg, or about 20 mg to about 100 mg, or about 25 mg to about 500 mg, or about 25 mg to about 400 mg, or about 25 mg to about 300, or about 25 mg to about 200 mg, or about 25 mg to about 150 mg, about 25 mg to about 100 mg, about 100 mg to about 500 mg, about 100 mg to about 400 mg, about 100 mg to about 300 mg, or about 100 mg to about 275 mg. In some embodiments, the SDD indibulin compositions described herein are administered at total daily doses of about 100 mg, about 110 mg, about 120 mg, about 130 mg, about 140 mg, about 150 mg, about 160 mg, about 170 mg, about 180 mg, about 190 mg, about 200 mg, about 225 mg, about 260 mg, about 275 mg, about 300 mg, about 325 mg, about 350 mg, about 275 mg, about 400 mg, about 425 mg, about 450 mg, about 475 mg, or about 500 mg, or any combinations of any of the individual dosage amounts set forth above.

In further embodiments, the SDD indibulin compositions described herein are administered at total daily doses of about 5 mg, about 10 mg, about 15 mg, about 20 mg, about 25 mg, about 30 mg, about 40 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 350 mg, about 400 mg, about 450 mg, or about 500 mg.

In some embodiments, the SDD indibulin compositions described herein are dosed once daily. In other embodiments, the SDD indibulin compositions are dosed 1-3 times daily. In further embodiments, the SDD indibulin compositions are dosed 1-2 times daily. In another embodiment, the SDD indibulin compositions are dosed in a treatment cycle for a set period of days. In one embodiment, the SDD indibulin compositions are dosed in a treatment cycle for at least about 3 days, about 5 days, about 8 days, about 10 days, about 15 days, about 20 days, about 30 days, about 2 months, about 3 months, about 6 months, about 1 year at a time or for a lifetime. In another embodiment, following a dosage period of a certain number of dosage days is a set number of days off in between dosage periods in which the subject is not given the compositions. In one embodiment, the set number of days off in between dosage periods is about 3 days, about 6 days, about 9 days, about 12 days, about 15 days, about 20 days, about 30 days, or about 40 days. In some embodiments, when the SDD indibulin composition is dosed once time per day, the plasma Cmax is greater than about 25 ng/mL, greater than about 30 ng/mL, greater than about 35 ng/mL, greater than about 40 ng/mL, greater than about 50 ng/mL, greater than about 60 ng/mL, greater than about 80 ng/mL, greater than about 100 ng/mL, greater than about 120 ng/mL, greater than about 140 ng/mL, or greater than about 160 ng/mL. In some embodiments, when dosed once per day at less than 100 mg/day, the plasma Cmax is greater than about 25 ng/mL, greater than about 30 ng/mL, greater than about 35 ng/mL, greater than about 40 ng/mL, greater than 45 ng/mL, or greater than 50 ng/mL. In some embodiments, when dosed once per day at about 25 mg/day to about 100 mg/day, the plasma Cmax ranges from about 25 ng/mL to about 200 ng/mL, about 25 ng/mL to about 150 ng/mL, about 45 ng/mL to about 120 mg/mL. In some embodiments, when dosed once per day at about 100 mg/day to about 275 mg/day, the plasma Cmax ranges from about 50 ng/mL to about 350 ng/mL, about 50 ng/mL to about 300 ng/mL, about 50 ng/mL to about 250 ng/mL, about 50 ng/mL to about 200 ng/mL, about 50 ng/mL to about 180 mg/mL, about 50 ng/mL to about 160 mg/mL, about 50 ng/mL to about 140 mg/mL, about 50 ng/mL to about 120 mg/mL, or about 50 ng/mL to about 100 mg/mL. In some embodiments, when dosed once per day at about 25 mg/day to about 350 mg/day, the plasma Cmax ranges from about 25 ng/mL to about 300 ng/mL, about 25 ng/mL to about 200 ng/mL, about 25 ng/mL to about 180 ng/mL, about 25 ng/mL to about 160 ng/mL, about 25 ng/mL to about 140 mg/mL, about 25 ng/mL to about 120 mg/mL, about 25 ng/mL to about 100 mg/mL. In some embodiments, when dosed once per day at about 25 mg/day to about 450 mg/day, the plasma Cmax ranges from about 25 ng/mL to about 400 ng/mL, about 25 ng/mL to about 300 ng/mL, about 25 ng/mL to about 200 ng/mL, about 25 ng/mL to about 180 ng/mL, about 25 ng/mL to about 160 ng/mL, about 25 ng/mL to about 140 mg/mL, about 25 ng/mL to about 120 mg/mL, about 25 ng/mL to about 100 mg/mL.

In certain embodiments, when the SDD indibulin composition is dosed once time per day, the SDD indibulin compositions described herein are dosed at about 100 mg to about 500 mg, about 100 mg to about 400 mg, about 100 mg to about 300 mg, or at about 100 mg, about 110 mg, about 120 mg, about 130 mg, about 140 mg, about 150 mg, about 160 mg, about 170 mg, about 180 mg, about 190 mg, about 200 mg, about 225 mg, about 260 mg, about 275 mg, about 300 mg, about 325 mg, about 350 mg, about 275 mg, about 400 mg, about 425 mg, about 450 mg, about 475 mg, or about 500 mg, or any combinations of any of the individual dosage amounts set forth above.

In some embodiments, when the SDD indibulin composition is dosed once time per day, the plasma AUC₁₋₂₄ is greater than about 200 hr·ng/mL, about 800 hr·ng/mL, or about 1000 hr·ng/mL. In some embodiments, when dosed once per day at less than 100 mg/day, the plasma AUC₁₋₂₄ is greater than about 100 hr·ng/mL, greater than about 125 hr·ng/mL, greater than about 150 hr·ng/mL or greater than about 200 hr·ng/mL. In some embodiments, when dosed once per day at about 25 mg/day to about 100 mg/day, the plasma AUC₁₋₂₄ ranges from about 100 hr·ng/mL to about 2000 hr·ng/mL, about 125 hr·ng/mL to about 1050 hr·ng/mL, or about 150 hr·ng/mL to about 1050 hr·ng/mL. In some embodiments, when dosed once per day at about 100 mg/day to about 275 mg/day, the plasma AUC₁₋₂₄ ranges from about 600 hr·ng/mL to about 3000 hr·ng/mL, about 600 hr·ng/mL to about 1600 hr·ng/mL, about 600 hr·ng/mL to about 1400 hr·ng/mL. In some embodiments, when dosed once per day at about 275 mg/day to about 450 mg/day, the plasma AUC₁₋₂₄ ranges from about 2000 hr·ng/mL to about 5000 hr·ng/mL, about 2000 hr·ng/mL to about 4000 hr·ng/mL, or about 2000 hr·ng/mL to about 3800 hr·ng/mL.

In an embodiment, the SDD indibulin compositions are dosed to the subject in a fasted state. In an embodiment, the subject is fasted overnight. As used herein, the term “fasted” means that the patient has not eaten any food (clear fluids only) for at least 2 hours, at least 4 hours, for at least 6 hours, for at least 8 hours, for at least 10 hours, or for at least 12 hours prior to administration of a provided formulation. In certain embodiments, the term “fasted” means an overnight fast.

In other embodiments, the compositions are administered to a subject that has not fasted. In another embodiment, the SDD indibulin compositions are dosed to the subject in a fed state. As used herein, the term “fed” means a standard meal (such as a full American breakfast) has been administered after an overnight fast of at least 10 hours and a meal starting 30 minutes prior to drug administration.

In one embodiment, the subject is dosed with the SDD indibulin compositions under a fed state, resulting in a higher plasma exposure level than when the subject is dosed with the SDD indibulin compositions under a fasted state. In one embodiment, the plasma exposure levels are about 1-5 fold higher when the SDD indibulin compositions are dosed to the subject in the fed state when compared to the fasted state. In another embodiment, the plasma exposure levels are about 1-2, about 1-3, about 1-4, about 2-3, about 2-4, about 2-5×, about 3-5, about 3-4, or about 4-5 fold higher when the SDD indibulin compositions are dosed to the subject in the fed state when compared to the fasted state.

In one embodiment, the subject is dosed with the SDD indibulin compositions under a fed state, resulting in a delayed Tₘₐₓ compared to when the subject is dosed with the SDD indibulin compositions under a fasted state.

### EXEMPLIFICATION

The invention now being generally described, it will be more readily understood by reference to the following examples which are included merely for purposes of illustration of certain aspects and embodiments of the present invention, and are not intended to limit the invention in any way.

Other objects, features, and advantages of the present invention will be apparent to those skilled in the art from a consideration of the foregoing detailed description and the following description of preferred exemplary embodiments thereof.

### Example 1

### Spry-Dried Solid Dispersion of Indibulin

### (a) Indibulin HPMCAS SDD Preparation and Physicochemical Characterization

General procedure: Solid spray-dried dispersions of indibulin and HPMCAS were prepared as follows. Crystalline indibulin and matrix polymer HPMCAS were dissolved in a 95:5 w/w tetrahydrofuran (THF) and water solution to form a spray solution. The THF and water were subsequently removed during spray-drying using a small-scale spray-drying apparatus (GEA-Niro Mobil Minor spray dryer), resulting in a homogenous dispersion in powder form. Using this method, 10 percent by weight active drug (% A) HPMCAS-H(HPMCAS high grade), 15% A HPMCAS-H, 20% A HPMCAS-H, 25% A HPMCAS-H, 25% A HPMCAS-M (HPMCAS medium grade) and 50% A HPMCAS-M SDD formulations were prepared. FIG. 2 is a flow chart providing an overview of a process used to manufacture the SDD of indibulin according to one embodiment on a GEA-Niro Mobil Minor spray dryer.

The mixture is then spray dried under appropriate conditions using a GEA-Niro Mobile Minor Spray Dryer With 6-foot and 9-inch Extension, DPH Gas Disperser, Cooling Fluid, and Nozzle-Centering Device; Pressure Nozzle: Steinen A75; Product Collection: 6-inch outer-diameter cyclone; solution filter less than or equal to 250 micron filter size. A 95:5 w/w THF:water solution was used for warmup and shutdown of the spray dryer. Following the spray drying process, the mixture was processed through a secondary drying step using a tray dryer (Gruenberg).

These SDDs were characterized by differential scanning calorimeter (DSC) and scanning electron microscope (SEM) and powder x-ray diffraction (PXRD). PXRD was taken using the methods and conditions summarized in Table 1 The characterization results are summarized in Table 2.

TABLE 1
Methods and Conditions

| Site | X-ray |
|---|---|
| Instrument | Bruker AXS |
| Detector | Scintillation counter |
| Scan Type | Detector scan |
| Scan Mode | Continuous scan |
| Scan Axis | 2Θ |
| Start | 4° |
| Stop | 40° |
| Step Size | 0.0400° |
| Time/Step | 2 sec |
| Total Scan Time | 30 min |
| Rotation | On |
| Voltage | 45 kV |
| Current | 40 mA |
| Holder | Zero background holder (ZBH) |
| Fixed Source Angle | 3.0° |

TABLE 2
Characterization of indibulin SDDs

| | T_{g} Based On | | Crystallinity |
|---|---|---|---|
| Sample | DSC (° C.) | SEM Observation | Based on PXRD |
| 10% A HPMCAS-H SDD | 102 | Whole, collapsed spheres | Amorphous |
| 15% A HPMCAS-H SDD | 96 | Whole, collapsed spheres | Amorphous |
| 20% A HPMCAS-H SDD | 92 | Whole, collapsed spheres | Amorphous |
| 25% A HPMCAS-H SDD | 87 | Whole, collapsed spheres | Amorphous |
| 25% A HPMCAS-M SDD | 89 | Whole, collapsed spheres | Amorphous |
| 50% A HPMCAS-M SDD | 74 | Whole, collapsed spheres | Amorphous |
| Bulk crystalline drug | NT^{a} | NT | Crystalline |

^{a}NT = Not Tested

### (b) Batch Manufacture of 10% A Indibulin HPMCAS SDD

Water (353.0 g) and THF (6706.0 g) were mixed in a stainless-steel solution tank. To the THF/water solution was added indibulin (65.6 g) and HPMCAS-HG (590.4 g), forming a spray solution. The resulted spray solution was subsequently spray-dried on a GEA-Niro Mobile Minor Spray Dryer under the conditions listed in Table 3. These conditions were divided into four sets: (A) preheating, (B) warmup, (C) feed-solution processing, and (D) shutdown.

TABLE 3
Spray-drying conditions for manufacture of 10% A indibulin
SDD on a GEA-Niro Mobile Minor Spray Dryer.

| | | | | | |
|---|---|---|---|---|---|
| | | Dryer | Dryer | | |
| | System Gas | Inlet | Outlet | Feed | |
| | Flow | Temp | Temp | Pressure | Feed Rate |
| Process | (FI-001) | (TIC-001) | (TIC-002) | (PI-007) | (FE/FI-002) |

Preheat

| | | | | | |
|---|---|---|---|---|---|
| Target | 1850 g/min | 110° C. | NA^{a} | NA | NA |
| Target | 1550-2150 | 100° C.- | NA | NA | NA |
| Range | g/min | 120° C. | | | |

Warmup

| | | | | | |
|---|---|---|---|---|---|
| Target | 1850 g/min | 110° C. | 48° C. | 650 psig | 155 g/min |
| Target | 1550-2150 | 100° C.- | 43-53° C. | 450-850 | 130-180 |
| Range | g/min | 120° C. | | psig | g/min |

Feed Solution Processing

| | | | | | |
|---|---|---|---|---|---|
| Target | 1850 g/min | 110° C. | 45° C. | 500 psig | 170 g/min |
| Target | 1550-2150 | 100° C.- | 40-50° C. | 400-600 | 155-185 |
| Range | g/min | 120° C. | | psig | g/min |

Shutdown

| | | | | | |
|---|---|---|---|---|---|
| Target | 1850 g/min | 110° C. | 48° C. | 650 psig | 155 g/min |
| Target | 1550-2150 | 100° C.- | 43-53° C. | 450-850 | 130-180 |
| Range | g/min | 120° C. | | psig | g/min |

^{a}NA = not applicable.

Bulk and tapped specific volumes and the particle size distribution of the SDD batch of 10% A Indibulin HPMCAS were determined according to the method described in United States Pharmacopoeia Chapter 616. The results are summarized in Table 4 and FIG. 3. Furthermore, methods for determining bulk and tapped specific volumes and the particle size of SDDs are described, for example in U.S. Pat. No. 7,780,988. Table 5 summarizes the volume frequency % at different particle diameters (micron). D(0.1) means that 10% of the particles in the sample have a diameter of 10 microns; D(0.5) means that 50% of the particles in the sample have a diameter of 32 microns; D(0.9) means that 90% of the particles in the sample have a diameter of 70 microns; D(3,2), which corresponds to the surface mean, is 20 micron; and D(4,3), which corresponds to the volume mean, is 37 microns. An SEM image of the SDD batch of 10% A Indibulin HPMCAS at 500× magnification is shown in FIG. 4.

TABLE 4
Bulk specific volume (BSV) and tapped specific volume
(TSV) of the SDD batch of 10% A indibulin HPMCAS-HG.

| | |
|---|---|
| BSV (cc/g) | 5.2 |
| TSV (cc/g) | 3.0 |

TABLE 5
Volume frequency % at different particle diameters (microns).

| | |
|---|---|
| D (0.1) (μm) | 10 |
| D (0.5) (μm) | 32 |
| D (0.9) (μm) | 70 |
| D (3.2) (μm) | 20 |
| D(4.3) (μm) | 37 |
| Span | 1.86 |

### Example 2

### In Vitro Dissolution and Suspension Stability of Indibulin HPMCAS SDD Formulations

### (a) Microcentrifuge Dissolution Test

The microcentrifuge dissolution test was performed to assess the ability of the indibulin SDD to sustain solubilized drug levels and to determine suspension stability. Using this method, 10% A HPMCAS-H(HPMCAS high grade), 15% A HPMCAS-H, 20% A HPMCAS-H, 25% A HPMCAS-H, 25% A HPMCAS-M (HPMCAS medium grade) and 50% A HPMCAS-M SDD formulations were tested. Specifically, the microcentrifuge dissolution test measures the supersaturation of drug above the crystalline solubility when dosed into the model fasted duodenal solution (MFDS).

In the microcentrifuge dissolution test, indibulin HPMCAS SDDs were each dosed into MFDS, which is 0.5% taurocholic acid, sodium salt hydrate (NaTC)/1-palmitoyl-2-oleoyl-sn-glycero-3-phosphocholine (POPC) in phosphate buffer solution (PBS, pH 6.5). In each resulted sample, the concentration of dissolved indibulin would have been 200 μg active drug per mL (200 μgA/mL), if all of the indibulin had dissolved. The samples were centrifuged at 13,000 rpm at 37° C. for 1 minute. The supernatant solution of each sample was then analyzed by HPLC. The remaining sample was mixed and allowed to stand undisturbed until next measurement was taken. Measurements were taken at 4, 10, 20, 40 and 90 minutes.

The 10% A, 15% A, 20% A, and 25% A HPMCAS-H SDDs showed substantially enhanced levels of solubilized drug as seen by maximum drug concentration (Cₘₐₓ) and an approximate 10-fold improvement in solubilized drug sustainment as seen by area under the curve through 90 minutes (AUC₉₀) when compared to bulk crystalline indibulin. Table 6 and FIG. 5 show these results. Table 6 is a summary of observed maximum concentrations of indibulin in MFDS for the first 90 minutes (Cₘₐₓ₉₀), area under the curve values for the first 90 minutes (AUC₉₀), and concentrations of indibulin in MFDS at 90 minutes and at 1200 minutes, following the introduction into MFDS.

TABLE 6
Summary of observed maximum concentrations
of indibulin in MFDS.

| | Cₘₐₓ₉₀ | AUC₉₀ | C₉₀ | C₁₂₀₀ |
|---|---|---|---|---|
| Sample | (μg/mL) | (min*μg/mL) | (μg/mL) | (μg/mL) |
| 10% A: HPMCAS-H | 36 | 1,870 | 13 | 10 |
| SDD | | | | |
| 15% A: HPMCAS-H | 35 | 1,570 | 11 | 8 |
| SDD | | | | |
| 20% A: HPMCAS-H | 36 | 1,380 | 9 | 8 |
| SDD | | | | |
| 25% A: HPMCAS-H | 37 | 1,250 | 8 | 8 |
| SDD | | | | |
| 25% A: HPMCAS-M, | 35 | 630 | 5 | 8 |
| SDD | | | | |
| 50%: HPMCAS-M, | 10 | 620 | 6 | 6 |
| SDD | | | | |
| Bulk Crystalline Drug | 2 | 160 | 1 | 3 |

Suspension stability of 10% A HPMCAS-H and 25% A HPMCAS-H formulations were evaluated using the microcentrifuge dissolution test, as detailed below.

First, a 0.5 wt % Methocel A (Methylcellulose, Methocel A4M Premium, DOW Chemical Company) vehicle was prepared and later used as the suspension vehicle. Specifically, this vehicle was prepared by adding 0.5 g of Methocel A to 30 mL deionized water at 90° C. and stirring until well dispersed. To the resultant dispersion was added 70 mL of water and the mixture was placed into an ice bath and stirred until Methocel A is dissolved.

A 20 mgA/mL indibulin SDD suspension of each of the 10% A HPMCAS-H and 25% A HPMCAS-H formulations was then prepared by adding the indibulin HPMCAS SDD to the aqueous 0.5 wt % Methocel A vehicle (as prepared above). Each suspension was then tested in the microcentrifuge dissolution test (as described above) immediately after constitution in the aqueous 0.5 wt % Methocel A vehicle (O-hour or Initial), and one hour after the constitution in the aqueous 0.5 wt % Methocel A vehicle. The stability of each suspension was determined by comparing the dissolution profile of the one-hour suspension to the performance of the initial suspension. The 10% A and 25% A HPMCAS-H SDDs both showed no change in performance after one hour. These results are shown in FIG. 6 and Table 7. Table 7 is a summary of observed maximum concentrations of indibulin for the first 90 minutes (Cₘₐₓ₉₀) and the dissolution area under the curve values for the first 90 minutes (AUC₉₀), following the introduction of the suspensions into MFDS.

TABLE 7
Summary of observed maximum concentrations
of indibulin in MFDS.

| | Suspension | | |
|---|---|---|---|
| | Hold Time | Cₘₐₓ₉₀ | AUC₉₀ |
| Sample | (hr) | (μg/mL) | (min*μg/mL) |
| Bulk Crystalline Drug | 0 | 2 | 150 |
| 10% A HPMCAS-H SDD | 0 | 40 | 1,740 |
| | 1 | 36 | 1,720 |
| 25% A HPMCAS-H SDD | 0 | 39 | 1,150 |
| | 1 | 37 | 1,080 |

### (b) Ultracentrifuge Test

In the ultracentrifuge test, the formulation (SDD or bulk crystalline indibulin) was added to a dissolution medium so that the concentration of dissolved indibulin would have been 200 μg active drug per mL (200 μgA/mL), if all of the indibulin had dissolved. The resulted mixture was then mixed for 90 minutes. The dissolution media used in this test are PBS (pH 6.5), 0.5% NaTC/POPC in PBS (pH 6.5), and 2% NaTC/POPC in PBS (pH 6.5). Following this step, indibulin/polymer colloidal species were then removed by ultracentrifugation at 80000 rpm for 20 minutes at 37° C. The concentration of the drug remaining in the supernatant was then analyzed by HPLC using conditions as specified in Table 8 below.

TABLE 8
Methods and conditions for analyzing
the ultracentrifuge test samples.

| | |
|---|---|
| Permeate | 80/20 (wt/wt) Cyclohexanone/Decane |
| Diluent | IPA |
| Concentration Range | 0.3 to 33 μg/mL |
| Sample Injection Volume | 10 μL |
| Column | Agilent Zorbax RX-SIL, 150 × 4.6 mm, 5 μm |
| Flow Rate | 1.0 mL/min |
| Run Time | 4 min |
| Retention Time | 2.6 min |
| Mobile Phase | 60:40 Hexane:IPA |
| Temperature | 40° C. |

Wavelength/Bandwidth

| | |
|---|---|
| Signal | 275 nm/8 nm |
| Reference | 550 nm/50 nm |

Free drug levels were measured for the 10% A HPMCAS-H, 15% A HPMCAS-H, 20% A HPMCAS-H, 25% A HPMCAS-H, 25% A HPMCAS-M and 50% A HPMCAS-M SDD formulations, at a dose of 200 ugA/mL in PBS (pH 6.5), 0.5% NaTC/POPC in PBS (pH 6.5), or 2% NaTC/POPC in PBS (pH 6.5) in the ultracentrifuge test, and the results are shown in FIG. 7(A).

The 10% A, 15%, and 20% A HPMCAS-H SDDs have an approximately 10-fold increase in free drug levels compared to the bulk crystalline indibulin drug in MFDS, while the HPMCAS-M SDDs and the 25% A HPMCAS-H SDD provided about a 5-fold increase in free drug solubility in this medium. In 2% NaTC/POPC, pH 6.5 dissolution media (simulating fed state conditions) the HPMCAS-H SDDs provide a 50- to 60-fold increase in free drug levels compared to bulk crystalline drug. The HPMCAS-M SDDs provide about a 20-fold enhancement in free drug levels compared to bulk crystalline drug in 2% NaTC/POPC, pH=6.5 media.

The micelle partition coefficient (Kₚ) of the bulk crystalline indibulin was also determined using ultracentrifugation at 37° C. in a temperature-controlled box. A typical procedure for determining the Kₚ value is described as follows:
1. Accurately weigh 0.36 mg active drug indibulin, or 3.6 mg of 10% A:HPMCAS-H SDD (or any other indibulin SDD) into 2.0 mL Sorenson microcentrifuge tubes. Prepare in duplicate for each micelle concentration.
   i. PBS at pH 6.5
   ii. 0.5-wt % NaTC/POPC in PBS at pH 6.5
   iii. 1.0-wt % NaTC/POPC in PBS at pH 6.5
   iv. 2.0-wt % NaTC/POPC in PBS at pH 6.5
2. Add 1.8 mL of the micelle solution using a Gilson 5-mL Pipetman to each tube
3. Start the timer.
4. Vortex the samples on Setting 8 on a Fisher Vortex Genie 2 for 1 minute.
5. Rock samples continuously.
6. At 90 minutes, centrifuge the samples for 1 minute at 13,000 rpm (15,800 g) using an IEC Micromax microcentrifuge.
7. Extract 250 μL of supernatant from each microcentrifuge tube using a Gilson 250-μL Pipetman and add the supernatant to one of the ultracentrifuge tubes.
8. Ultracentrifuge for 8 minutes at 37° C., 80,000 RPM (300,000 g) using a Beckman Optima MAX.
9. Add 250 μL of diluent into HPLC vials using a Gilson 250-μL Pipetman. Extract 50 μL of supernatant from each ultracentrifuge tube using a Gilson 50-μL Pipetman and add the supernatant to one of the HPLC vials containing 250 μL of diluent.
10. Cap and crimp the HPLC vials and invert twice to mix
11. Vortex for 25 seconds.
12. Repeat Steps 5 through 10 with the sample when the timer reads 24 hours.
Note: Start the microcentrifuge approximately 90 seconds before each sample is taken.
13. Analyze the samples by HPLC.
14. Dynamic light scattering (DLS) is used to determine the presence of colloids or agglomerations in solution before and after ultracentrifugation. A sample of at least 50 μL is taken before and after ultracentrifugation for comparison.

Kₚ can be calculated using the equation: [Dₜ/D_{f}]=Vₘ*Kₚ+1, where: Dₜ=Average drug concentration (mg/mL); Vₘ=Wt % micelles in solution; Kₚ=Micelle partition coefficient, D_{f}=Drug concentration in aq. solution (mg/mL) (i.e. PBS, no micelles), (1-Vₘ)=Aq. volume fraction (the volume of micelles is very small compared to the volume of aqueous solution so (1-Vₘ) can simply be 1. FIG. 7(B) shows the partition coefficient result of bulk crystalline indibulin using the above method, where Kₚ is the slope of the Dₜ/D_{f} vs. Vₘ. The Kₚ for any indibulin SDD can also be determined using the same procedure.

### (c) Membrane-Permeation Test

The 10% A HPMCAS-H(HPMCAS high grade), 15% A HPMCAS-H, 20% A HPMCAS-H, 25% A HPMCAS-H, 25% A HPMCAS-M (HPMCAS medium grade) and 50% A HPMCAS-M SDD formulations were also evaluated in a membrane-permeation test at 37° C. in a temperature-controlled box. Test procedures were described, for example, in U.S. Pat. No. 7,611,630. For example, a membrane-permeation test of 10% A HPMCAS-H indibulin SDD was performed using the following procedures:
1. Accurately weigh 1 mgA (10 mg of 10% A:HPMCAS-H SDD) into the outer-glass reservoir of the test apparatus. All samples are prepared in duplicates.
2. Add 5 mL MFDS and start the timer.
3. Cap each reservoir and vortex at medium setting for 1 minute. Stir at 100 rpm.
4. After 9 minutes of addition of MFDS, add 5 mL of 80/20 (wt/wt) Cyclohexanone/Decane permeate to the inside of the membrane cells.
5. After 1 minute (at 10 minutes) place a cell containing the permeate solution into the outer reservoir holding the feed/drug solution. Take a O-minute time point. Tilt the entire apparatus at a 45° angle to ensure that no air bubbles are trapped under the membrane.
6. Place apparatus back on stir plate at 100 rpm.
7. Cover the apparatus with an inverted 100-mL beaker to limit sample evaporation.
8. Approximately 20 seconds before each sampling time point, add 250 mL of isopropyl alcohol (IPA) to the amber HPLC vials.
9. Remove 50-μL aliquots from the permeate solution at 0, 15, 30, 45, 60, 90, 120, 180, and 240 minutes and add to IPA in amber HPLC vials, and analyze the samples by HPLC. The same test was performed using 15% A HPMCAS-H, 20% A HPMCAS-H, 25% A HPMCAS-H, 25% A HPMCAS-M (HPMCAS medium grade) and 50% A HPMCAS-M SDD indibulin formulations.

A typical membrane permeation set-up that can be used in the above membrane permeation test is shown in FIG. 8.

This test assessed the rate of solubilized drug indibulin that passed through a synthetic membrane from aqueous MFDS to an aqueous immiscible organic sink solution. The SDD formulations and the bulk crystalline indibulin all had relatively low fluxes in the membrane-permeation test, with the 10% A HPMCAS-H SDD having the highest flux and drug recovery. FIG. 9 and Table 9 show these results. Table 9 is a summary of the observed total drug recovery (% dose) and average flux through the membrane (μgA/cm²×min).

TABLE 9
Observed total drug recovery (% dose) and average flux
through the membrane (μgA/cm² × min).

| | Total Drug Recovery | Average Flux |
|---|---|---|
| Sample | (% Dose) | (μgA/cm² × min) |
| Crystalline Indibulin | 10 | 0.10 |
| 10% A HPMCAS-H SDD | 24 | 0.20 |
| 15% A HPMCAS-H SDD | 17 | 0.16 |
| 20% A HPMCAS-H SDD | 15 | 0.13 |
| 25% A HPMCAS-H SDD | 11 | 0.09 |
| 25% A HPMCAS-M SDD | 10 | 0.08 |
| 50% A HPMCAS-M SDD | 7 | 0.06 |

### Example 3

### Physical and Chemical Stability Studies of Indibulin HPMCAS SDDs

The glass transition temperature (T_{g}) of the SDD formulations 10% A HPMCAS-H (HPMCAS high grade), 15% A HPMCAS-H, 20% A HPMCAS-H, 25% A HPMCAS-H, 25% A HPMCAS-M (HPMCAS medium grade) and 50% A HPMCAS-M SDD is determined as a function of relative humidity (RH) as shown FIG. 10(A).

Typically, T_{g}s of the SDD formulations were determined using a modulated differential scanning calorimetry (MDSC) method using the conditions as specific in Table 10 below. A representative MDSC Thermogram of the 10% A HPMCAS-H indibulin SDD is shown in FIG. 10(B).

TABLE 10
Method and conditions for the MDSC method to determine T_{g}.

| | |
|---|---|
| Instrument | TA Q1000 MDSC |
| Sample Mass | 5 mg (±2 mg) |
| Sample Preparation | 5-mm pellet, <5% RH overnight (>15 hr) |
| Pan | TA standard pan |
| Scan Range | −20° C. to 180° C. |
| Scan Rate | 2.5° C./min |
| Modulation | ±1.5° C./min |
| Nitrogen Flow | 50 mL/min |
| Data Sampling Interval | 0.20 sec/point |
| Load Temperature | 15° C. to 25° C. |
| T_{g} Calculation | Half height |

The results demonstrated that the SDDs have T_{g}s that are sufficiently high to ensure long-term physical stability of the SDD if protected from high humidity.

Samples of each formulation were also exposed for 24 hours to high temperature and humidity (i.e., 50° C./75% RH). These samples were then analyzed by SEM to determine any physical changes that occurred during storage. Potency was measured by high-performance liquid chromatography (HPLC) to evaluate chemical changes. These results are shown in Table 11. No significant changes in potency were observed. All indibulin SDDs with a drug loading of 25% or lower were free of crystals and particle fusing. Crystals, however, were observed by SEM on the 50% A HPMCAS-M SDD. This result was expected because the T_{g} and the storage temperature for this SDD are the same at 50/75% RH, which likely allows sufficient mobility and crystallization of the drug within the polymer matrix.

TABLE 11
Potency and SEM observations of Indibulin SDD stability samples
before and after exposure to 50° C./75% RH for 1 day.

| | Potency (mgA/g) | | | |
|---|---|---|---|---|
| Indibulin | | 1 day at 50° | SEM Observations | |
| Formulation | Initial | C./75% RH | Fusing | Crystals |
| 10% A HPMCAS-H SDD | 97 | 95 | No | No |
| 15% A HPMCAS-H SDD | 148 | 148 | No | No |
| 20% A HPMCAS-H SDD | 196 | 195 | No | No |
| 25% A HPMCAS-H SDD | 242 | 245 | No | No |
| 25% A HPMCAS-M SDD | 249 | 247 | No | No |
| 50% A HPMCAS-M SDD | 488 | 488 | No | Yes |

### Example 4

### An Indibulin SDi Tablet Formulation

A 25 milligrams active drug (mgA) indibulin spray-dried intermediate (SDi) tablet formulation was developed. The composition of the tablet formulation is summarized in Table 12.

TABLE 12
Composition of the 25 mgA indibulin SDi tablet,
which contains 10% A indibulin HPMCAS-H SDD.

| | | | | |
|---|---|---|---|---|
| | | | Composition | mg/ |
| Component | Function | Grade | (% w/w) | tablet |

Active Ingredient

| | | | | |
|---|---|---|---|---|
| Indibulin: HPMCAS -H | Active | NA^{c} | 50.00 | 250.0 |
| SDi 100 mg/gb | | | | |

Intragranular Excipients

| | | | | |
|---|---|---|---|---|
| Microcrystalline | Filler | NF^{d} | 22.50 | 112.50 |
| Cellulose | | | | |
| (Avicel PH101) | | | | |
| Lactose Monohydrate | Filler | NF | 22.50 | 112.50 |
| (Regular 310) | | | | |
| Croscarmellose Sodium | Disintegrant | NF | 3.00 | 15.00 |
| (Ac-Di-Sol) | | | | |
| Colloidal Silicon | Glidant | NF | 1.0 | 5.00 |
| Dioxide (CabOSil M5P) | | | | |
| Magnesium Stearate | Lubricant | NF | 0.25 | 1.25 |

Extragranular Excipients

| | | | | |
|---|---|---|---|---|
| Colloidal Silicon | Glidant | NF | 0.50 | 2.50 |
| Dioxide (CabOSil M5P) | | | | |
| Magnesium Stearate | Lubricant | NF | 0.25 | 1.25 |

| | | |
|---|---|---|
| Total core tablet weight (mg) | 100.00 | 500.00 |

^{c}NA = not applicable.
^{d}NF = National Formulary.
e. Removed during the coating process to minimal levels, does not add to the weight of the
tablet
f. United States Pharmacopoeia

The 25 mgA indibulin SDi tablets were prepared following the procedures as described in FIG. 11. Tables 13-15 below list exemplary Gerteis Mini-Pactor Roller-compactor parameters, tablet compression parameters and in-process controls for making the 25 mgA indibulin SDi tablets using 10% A HPMCAS-H indibulin SDD. A 3-10× portion of the SDD indibulin was added to colloidal silicon dioxide and mixed manually for 15-30 seconds and passed through a 20-mesh screen into a PK Blend Master Lab Blender. A bulk density of 0.30 g/cc was used. In the first blending step, a 12 rpm speed was used for about 10 minutes. For the second blending step, microcrystalline cellulose, lactose monohydrate, and croscarmellose sodium were added and blended for 12 rpm for about 15 minutes. Following the blending steps, a Quadro Comil Model 197 was used for delumping and was affixed with a 032R screen size, a 1601 mill impellor, at about 1500 rpm. A 3-10× portion of the blend mixture was combined with magnesium stearate lubricant and manually mixed for about 30 seconds. After passing the blend mixture with the lubricant through a 20 mesh screen into the blender, the entire mixture was blended at 12 rpm for about 5 minutes, resulting in a pre-granulation blend.

During the granulation step, a Gerteis Mini-Pactor Roller was used to roller compact the pre-granulation blend into ribbons (see Table 13 for parameters). Comil Model 197 was then used with a 050G screen size for producing a granulation mixture to a target solid fraction of 0.65 (±0.02) using a true density of 1.400 g/cm³.

The PK Blend Master Lab Blender with a 20 mesh screen size with was used for the final blending step. A bulk density of 0.40 g/cc was used. Colloidal silicon dioxide was blended with a portion of the granulation mixture manually and passed through the screen. The resulting mixture was then returned to the blender and blended at 12 rpm for about 15 minutes. Afterwards, magnesium stearate lubricant was manually mixed with a portion of the blender mixture and passed through a 20 mesh screen. The resulting mixture was then returned to the blender and blended at 12 rpm for about 5 minutes.

A Korsch XL100 tablet press was used according to the parameters in Table 14.

The 50 mgA indibulin tablets were prepared using procedures similar to those described above for the 25 mgA tablets with appropriate adjustment of ingredient amount and process parameters.

TABLE 13
Gerteis Mini-Pactor Roller-Compactor Parameters
For Manufacture Of 25-mgA Indibulin SDi Tablets

| Parameter | Setting |
|---|---|
| Compression force (kN/cm) | Adjust as needed to |
| | achieve target solid |
| Roll speed (rpm) | 2 (±2) |
| Tamp/feed ratio (%) | 180 (±50) |
| Gap-control activated (yes/no) | Yes |
| Gap width (mm) | 2.0 (±0.5) |
| Torque control (on/off) | Off |

TABLE 14
Tablet Compression Parameters To Manufacture
25 mgA Indibulin SDi Tablets

| Parameter | Value |
|---|---|
| Tooling size | 0.3125 × 0.6250 oval concave face |
| Tooling drawing number | P13361-97B |
| Number of tooling stations^{a} | 4 |
| Turret speed^{a} | 20 rpm or sufficient for press run |
| | time to exceed 45 minutes |
| Tablet weight | 500.0 mg |
| Fill cam depth^{b} | estimated 8.5-16 mm |
| Feed frame speed | Minimum needed to maintain |
| | tablet weight |
| Tablet hardness | 14-18 kP |
| Precompression force^{b} | As needed to achieve tablet hardness |
| Approximate tablet thickness^{b} | 6.3 mm |
| Deduster | Yes |
| Metal checker | Yes |

^{a}Recommended or expected valued based on development experience; may need to be adjusted to meet local procedures and to maintain tablet characteristics within specifications.
^{b}Values used during development and provided for set-up guidance; actual compression force and resultant tablet thickness may need to be varied to meet target weight and hardness specifications.

TABLE 15
Proposed In-Process Controls for 25-mgA Indibulin SDi Tablets

| | | | |
|---|---|---|---|
| | Sample Time and | Test | Preliminary |
| Test | Quantity | Method | Acceptance Criteria |

Tablets

| | | | |
|---|---|---|---|
| Mean | Start-up and end | Analytical | 500.0 mg |
| tablet | (n = 100); and | balance | ±5% (working limit) |
| weight | during run at timed | BRPPD | ±7% (alert limit) |
| | sample points | SOP-3-027^{a} | |
| | (n = 10 tablets) | | |
| Tablet | n = 100 tablets | Analytical | Relative standard |
| weight | at start-up and end | balance | deviation (RSD) <3% |
| variation | | BRPPD | |
| | | SOP-3-027 | |
| Tablet | n = 3 tablets at | Crushing | 16 kP |
| hardness | timed sampling points | strength | ±2 kP (working limit) |
| | n = 10 at end of run | BRPPD | ±3 kP (alert limit) |
| | | SOP-3-027 | |
| Tablet | n = 20 tablets at | Friability | <0.3% |
| friability | timed sampling points | BRPPD | |
| | | SOP-2-027 | |
| Tablet | n = 3 tablets at | Caliper | Report results |
| thickness | timed sampling points | BRPPD | |
| | | SOP-3-027 | |
| Visual | Per acceptable | Appearance | White to off white, |
| inspection | quality limit | BRPPD | modified oval tablet |
| | (AQL) SOP | SOP-3-027 | AQL = 1.5 for minor |
| | | | defects, 0.40 for major |
| | | | defects, and 0.01 for |
| | | | critical defects |

^{a}SOP = standard operating procedure.

### Example 5

### Rat Pharmacokinetic Studies Using Indibulin SDDs

In order to obtain an improved formulation of indibulin with enhanced solubility, improved bioavailability and decreased variability in plasma pharmacokinetics, a SDD formulation of indibulin was developed. Several different formulations and active strengths were tested in vitro for performance, long term stability and manufacturability. Solubility and dissolution parameters were compared with those obtained with the crystalline indibulin. See, for example, FIG. 5 (B). Based on data obtained, two formulations, i.e., 10% A (10% active) and 25% A (25% active) HPMCAS-H SDD formulations were selected to be tested for bioavailability in rats. As detailed below, two studies were performed.

First Study: Indibulin Rat Plasma Concentration after Dosing with 10% A and 25% A Indibulin HPMCAS-H SDDs

In the first study, the crystalline indibulin, 10% A and 25% A SDDs were administered p.o. at 10 and 15 mg/kg to rats (Sprague Dawley, male) and plasma concentrations of indibulin were determined at several time points.

In particular, all rats used in this study were fasted 12 hours prior to dose, and food was returned 4 hours post dose. 10% A and 25% A indibulin HPMCAS SDD formulations (as prepared in Example 1(a)) were used in this study. Indibulin in crystalline form was suspended in 0.5% Methocel A at 10 mg/kg, and 15 mg/kg by vortexing and sonicating. The indibulin SDD formulations were made by combining 10% active indibulin SDD at 10 mg/kg and 15 mg/kg, 25% active indibulin SDD at 10 mg/kg and 15 mg/kg using a mortar with 0.5% Methocel A and using a pestle to gradually mix in to solution. There were six rats per group, all animals were weighed and dose volume calculated so that each rat received test article orally at a volume of 10 mL/kg. The first three rats in each group were bled at 0.5, 2, and 6 hours post dose; the remaining three rats in each group were bled at 1, 4, and 10 hours post dose. For blood collection at all time points all animals were anesthetized with an inhalation of 4% isoflurane and 1.5% oxygen, once it was determined that the animal was adequately anesthetized (no reaction to physical stimulation) using a capillary tube the orbital venous plexus was punctured and a volume of 1 mL of blood was collected in to a sodium heparin tube. Once the blood was collected the sodium heparin tube was inverted ten times, and placed on ice until centrifuged for 10 minutes at 2510 rpm. The plasma was decanted in to labeled Eppendorfs and stored at −80° C. until analyzed by HPLC with MS/MS Detection. All data were acquired using Applied Biosystems/MDS-Sciex Analyst Version 1.5, processed; reported using Watson Version 7.3.0.01TM, Thermo Fisher Scientific, Inc.; and are summarized in FIGS. 12 and 14 and Tables 16, 17, and 18. Table 16 is a summary of plasma indibulin concentrations at various times post dosing. Table 17 is summary of maximum plasma concentrations of indibulin (Cₘₐₓ), time for maximum plasma concentration to occur (tₘₐₓ), and plasma area under the curve values (AUC₀₋ₜ). Table 18 is a summary of the plasma pharmacokinetic data in rate at different doses (10 mg/kg) for two SDD formulations (10% and 25%, respectively), in comparison with the same doses for crystalline indibulin. Table 18 also includes comparative data for a solutol/propylene glycol indibulin formulation, administered by IV and orally.

TABLE 16
Summary of first and second PK rat study with SDD formulations.

| | | | | |
|---|---|---|---|---|
| | 10 mg/kg | 15 mg/kg | 20 mg/kg | |
| | crystalline | crystalline | crystalline | 10 mg/kg |
| Hours | indibulin | indibulin | indibulin | 10% 2nd |

ng/mL plasma indibulin

| 0 | 0 | 0 | 0 | 0 |
|---|---|---|---|---|
| 0.5 | 2 | 10 | 9 | 244 |
| 1 | 4 | 17 | 12 | 282 |
| 2 | 9 | 17 | 14 | 157 |
| 4 | 4 | 24 | 21 | 88 |
| 6 | 7 | 7 | 7 | 66 |
| 10 | 2 | 19 | 6 | 47 |
| | 15 mg/kg | 20 mg/kg | 10 mg/kg | 15 mg/kg |
| Hours | 10% 2nd | 10% 2nd | 10% 1st | 10% 1st |

ng/mL plasma indibulin

| | | | | |
|---|---|---|---|---|
| 0 | 0 | 0 | 0 | 0 |
| 0.5 | 273 | 390 | 190 | 239 |
| 1 | 262 | 401 | 213 | 319 |
| 2 | 215 | 256 | 161 | 184 |
| 4 | 122 | 191 | 72 | 92 |
| 6 | 91 | 165 | 127 | 95 |
| 10 | 73 | 146 | 48 | 37 |

TABLE 17
Maximum plasma concentrations of indibulin (Cₘₐₓ), time for maximum plasma
concentration to occur (tₘₐₓ), and plasma area under the curve values (AUC₀₋ₜ).

| | | | Second Study | | | First Study | | |
|---|---|---|---|---|---|---|---|---|
| Indibulin | Dose | Dose | Cₘₐₓ | tₘₐₓ | AUC₀₋ₜ | Cₘₐₓ | tₘₐₓ | AUC₀₋ₜ |
| formulation | (mg/m²) | (mg/kg) | (ng/ml) | (hours) | (ng * h/ml) | (ng/ml) | (hours) | (ng * h/ml) |
| crystalline | 60 | 10 | 8.6 | 2 | 48 | 18 | 2 | 61 |
| | 90 | 15 | 24 | 4 | 146 | 6 | 1 | 31 |
| | 120 | 20 | 21 | 4 | 110 | | | |
| 10% A | 60 | 10 | 282 | 1 | 1036 | 213 | 1 | 1117 |
| SDD | 90 | 15 | 273 | 0.5 | 1319 | 319 | 15 | 1178 |
| | 120 | 20 | 401 | 1 | 2053 | | | |

TABLE 18
Indibulin Plasma Pharmacokinetics in Rats (Summary)

| | Male Wistar Rats | | Male S-D Rats | | | | | |
|---|---|---|---|---|---|---|---|---|
| | Route | | | | | | | |
| | IV | Oral (Gavage) | Oral (Gavage) | | | | | |
| | Formulation | | | | | | | |
| | Solutol/Propyleneglycol | | | | | | | |
| | (3:1) | | Crystalline | | 10% SDD | | 25% SDD | |
| | Dose (mg/kg) | | | | | | | |
| | 0.25 | 10 | 10 | 15 | 10 | 15 | 10 | 15 |
| Terminal | 0.75 | 14.6 | ND | 5.9 | 9.6 | 3.7 | ND | 16.8 |
| t_{1/2} (hr) | | | | | | | | |
| Tₘₐₓ (hr) | 0.083 | 2 | 2 | 1 | 1 | 1 | 1 | 1 |
| Cₘₐₓ | 89.8 | 97.4 | 18 | 16.5 | 213 | 319 | 142 | 177 |
| (ng/mL) | | | | | | | | |
| AUC₀₋₁₀ | 65.1^{a} | 670^{b} | 63.6 | 56.6 | 956 | 1177 | 684 | 694 |
| (hr · ng/mL) | | | | | | | | |
| F (%)^{c} | 100% | 26% | 2.4% | 1.50% | 37% | 30% | 26% | 18% |

^{a}AUC_{0-1.5}, quantifiable concentrations up to 1.5 hr after IV dosing at 0.25 mg/kg
^{b}Partial AUC truncated from AUC₀₋₂₄ for comparison purpose; AUC₀₋₂₄ = 1204 hr · ng/mL
^{c}Calculated based on ratio of dose-normalized AUC₀₋₁₀Â after oral and IV administration; AUC₀₋∞ cannot be determined due to inadequate sampling time on the terminal phase

The maximum plasma indibulin (Cₘₐₓ) occurred at about 1 hour post administration for all formulations tested. At 15 mg/kg dosage, the average Cₘₐₓ for the 10% A formulation was about 319 ng/ml, for the 25% A formulation, the average Cₘₐₓ was 177 mg/ml, both of which are significantly higher than the 13 ng/ml Cₘₐₓ for crystalline indibulin.

The ratio of Cₘₐₓ of the 10% SDD solution to the Cₘₐₓ of the crystalline formulation is 11.9 and 19.4, for the 10 mg/kg and 15 mg/kg doses, respectively. The ratio of AUC of the 10% SDD solution to the AUC of the crystalline formulation is 15.0 and 20.8, for the 10 mg/kg and 15 mg/kg doses, respectively. These data confirm plasma exposure that are at least about 12-21 times higher with the 10% SDD indibulin, compared to crystalline indibulin. Moreover, the absolute oral bioavailability was 37% for SDD indibulin compared to 2.4% for crystalline indibulin. These improvements in plasma exposure are unexpected in view of predictive models, which predicted a more modest improvement in drug absorption, if any.

Second Study: Dose Response of Indibulin Rat Plasma Concentration after Dosing with 10% A Indibulin SDD

The second study was designed to determine the dose response of indibulin plasma concentration with the 10% A SDD formulation. In general, 10% A SDD was administered p.o. at 10, 15 and 20 mg/kg and plasma indibulin concentrations were determined at several time points.

All rats used in the second study were fasted 12 hours prior to dose, and food was returned 4 hours post dose. Indibulin in crystalline form was suspended in 0.5% Methocel A at 10 mg/kg, 15 mg/kg and 20 mg/kg by vortexing and sonicating. The indibulin SDD formulations were made by combining 10% active indibulin SDD at 10 mg/kg, 15 mg/kg and 20 mg/kg in a mortar with 0.5% Methocel A and using a pestle to gradually mix in to solution. There were six rats per group, all animals were weighed and the dose volume calculated so that each rat received the test article orally at a volume of 10 mL/kg. The first three rats in each group were bled at 0, 5, 2, and 6 hours post dose; the remaining three rats in each group were bled at 1, 4, and 10 hours post dose. For blood collection at all time points, all animals were anesthetized with an inhalation of 4% isoflurane and 1.5% oxygen; once it was determined that the animal was adequately anesthetized (no reaction to physical stimulation) using a capillary tube the orbital venous plexus was punctured and a volume of 1 mL of blood was collected in to a sodium heparin tube. Once the blood was collected the sodium heparin tube was inverted ten times, and placed on ice until centrifuged for 10 minutes at 2510 rpm. The plasma was decanted into labeled Eppendorfs and stored at −80° C. until analyzed by HPLC with MS/MS Detection. All data were acquired using Applied Biosystems/MDS-Sciex Analyst Version 1.5; processed, and reported using Watson Version 7.3.0.01TM, Thermo Fisher Scientific, Inc.; and are summarized in FIG. 13 and FIG. 14.

The result demonstrated that there was a marked dose response, in particular, an clear increase in indibulin plasma concentration with increasing doses, when the 10% A SDD was orally administered. Furthermore, the Cₘₐₓ for the 10% A SDD is about 33-fold higher than that of crystalline indibulin at 20 mg/kg dose, signifying a much improved bioavailability.

### Example 5

### Phase I/II Clinical Trial Study

A phase I study was conducted to determine the maximum tolerated dose (MTD) of indibulin SDD tablets when administered to subjects in an open-label, single-arm, multi-center study for 5 days followed by a 9 day rest (5-9) using a standard, 3+3 dose escalation scheme. Additional objectives of the study included evaluating the safety and pharmacokinetics (PK) of indibulin as well as describing the overall toxicity rates during a 28 day treatment cycle and estimating the overall response rate and the proportion of subjects who are progression-free at 4 months.

Key eligibility criteria for inclusion of subjects in the study were histologic or cytologic confirmation of carcinoma of the breast; metastatic disease or locally advanced disease not amenable to curative therapy; measurable or non-measurable lesions; any number of prior endocrine, biologic or chemotherapy regimens is permitted; prior radiation therapy is permitted; ECOG performance status of (0-2); age≧18 years; life expectancy≧12 weeks; adequate bone marrow, liver and renal function (Creatinine≦1.5×upper limit of normal (ULN); total bilirubin≦1.5×ULN; ALT or AST≦2.5×ULN; ANC≧1.5×109/L; Platelets≧100×109/L; Hemoglobin≧9 g/dL); and women of child bearing potential must use appropriate contraception.

Subjects excluded from the study included those with untreated symptomatic CNS metastases; uncontrolled gastrointestinal malabsorption; ≧Grade 2 peripheral neuropathy; history of an invasive second primary malignancy diagnosed within the

previous 3 years (except endometrial or cervical carcinoma or prostate carcinoma treated surgically; or non-melanoma skin cancer for Stage I endometrial or cervical carcinoma or

prostate carcinoma treated surgically, and non-melanoma skin cancer); any medical psychological or social condition that may interfere with the subject's ability to safely participate in the study; pregnant or nursing women.

Dose limiting toxicity (DLT) observation period was during days 1 to 15 of cycle 1. Serial blood collection was used for pharmacokinetic evaluation (see example 7). Tumor assessments for efficacy was conducted at baseline, at 8 week intervals for the first 6 months, and then every 12 weeks thereafter. Subjects were treated until disease progression or unacceptable toxicity. Adverse events (AEs) were graded by CTCAE v 4.0. Objective disease status was evaluated according to RECIST 1.1.

The phase I dose escalation cohorts was also used for a cross-over study for food-effect PK assessment. Cohorts 1 to 7 were subjects treated with a total dose (once daily) of 25 mg fasted, 50 mg fasted, 100 mg fasted, 150 mg fasted, 200 mg fasted, 275 mg fasted and fed, and 350 mg fed, respectively. The 25 subjects included 24 female and 1 male subjects with a median age of 58 (range 32-81), a median number of prior therapies of 5 (range 1-12), and an ECOG status of 0 (5 subjects), 1 (18 subjects), 2 (2 subjects). The majority of related toxicities are GI-related, mild to moderate in serverity and reversible. The most frequent and related AEs include decreased apetite, diarrhea, nausea, vomiting, and fatigue. No grade 3, 4, 5, or serious adverse events (SAEs) were reported as related to study medication. Two subjects had prolonged stable disease (SD). Specifically, one subject was progression free beyond 4 months after administration of 275 mg fasted (stable disease after cycle 6 of study) and one subject was progression free up to 4 months after administration of 150 mg fasted. The elimination of half-life of indibulin ranged from 6-12 hours.

### Example 6

### Human Pharmacokinetic Data from Phase I/H Clinical Trial Study

Crystalline indibulin capsules and indibulin SDD tablets were administered p.o. to breast cancer patients. The crystalline indibulin capsules were dosed at 600 mg and 800 mg, three times per day (TID) for a total daily dose of 1800 mg/day and 2400 mg/day. The SDD indibulin tablets were dosed under fasted conditions (i.e., clear fluids only from midnight until 2 hours post-dose) once per day (QD) for a total daily dose of 25 mg/day, 50 mg/day, 100 mg/day, 150 mg/day, 200 mg/day, and 275 mg/day (normalized to body surface area (BSA in m²) ranging from 14-177 mg/m²). Thus, total daily dose for the SDD tablet formulation was between 1-6% of the total daily dose for the crystalline capsules. The SDD indibulin tablets were also dosed under fed conditions once per day (QD) for a total daily dose of 275 mg/day (normalized to BSA ranging from 164-182 mg/m²) and 350 mg/day (normalized to BSA ranging from about 199-224 mg/m²). For each study, plasma concentrations of indibulin were determined. The data under fasted conditions are shown in Table 19, which is a summary of the plasma pharmacokinetic data for the two different indibulin formulations, at different doses. The data under fed conditions are shown in Table 20, which is a summary of the plasma pharmacokinetic data for the two different doses of indibulin SDD.

TABLE 19
Indibulin Plasma Pharmacokinetics in Patients (fasted conditions).

| | Breast Cancer Patients (Mean ± SD) | | | | | | | |
|---|---|---|---|---|---|---|---|---|
| | Formulation | | | | | | | |
| | Indibulin Crystaline (Capsules) | | SDD Formulated Indibulin (Tablets) | | | | | |
| | Dose (mg/day) | | | | | | | |
| | 1800 | 2400 | 25 | 50 | 100 | 150 | 200 | 275 |
| | (n = 3) | (n = 4) | (n = 5) | (n = 3) | (n = 3) | (n = 2)* | (n = 3) | (n = 3) |
| Dosing | TID, | TID, 800 mg, q5h | QD | QD | QD | QD | QD | QD |
| Regimen | 600 mg, q5h | | | | | | | |
| Tmax | 8.33 ± 2.89 | 7.25 ± 3.86 | 1.60 ± 0.55 | 1.67 ± 0.58 | 2.67 ± 1.15 | 4.0 | 3.3 ± 2.3 | 2.0 ± 0.0 |
| (hr) | | | | | | | | |
| Cmax | 122 ± 102 | 178 ± 83 | 49.5 ± 34.0 | 57.4 ± 26.2 | 120 ± 51 | 119 | 165 ± 89 | 158 ± 145 |
| (ng/mL) | | | | | | | | |
| AUC₀₋₂₄ | 774 ± 631 | 1045 ± 530 | 238 ± 82 | 429 ± 182 | 1025 ± 298 | 1163 | 1411 ± 111 | 1508 ± 1281 |
| (hr · ng/mL) | | | | | | | | |
| Cₘₐₓ | 0.068 ± 0.057 | 0.074 ± 0.035 | 1.98 ± 1.36 | 1.15 ± 0.52 | 1.20 ± 0.51 | 0.80 | 0.82 ± 0.45 | 0.57 ± 0.53 |
| (ng/mL)/ | | | | | | | | |
| Dose (mg) | | | | | | | | |
| AUC₀₋₂₄ | 0.430 ± 0.351 | 0.435 ± 0.221 | 9.5 ± 3.3 | 8.6 ± 3.6 | 10.3 ± 3.0 | 7.8 | 7.1 ± 0.6 | 5.5 ± 4.7 |
| (hr · ng/mL)/ | | | | | | | | |
| Dose (mg) | | | | | | | | |

*One subject had grade 2 pneumonia and received Morphine Sulphate on Day 1 of indibulin treatment; PK data from this subject was excluded due to impact of morphine on PK

At 25 mg/day, the average Cₘₐₓ for the SDD formulation was about 50 ng/ml, at 50 mg/day, the average Cₘₐₓ for the SDD formulation was about 57 ng/ml, at 100 mg/day, the average Cₘₐₓ for the SDD formulation was about 120 ng/ml, at 150 mg/day, the average Cₘₐₓ for the SDD formulation was about 119 ng/ml, at 200 mg/day, the average Cₘₐₓ for the SDD formulation was about 165 ng/ml, and at 275 mg/day, the average Cₘₐₓ for the SDD formulation was about 158 ng/ml. In comparison, the average Cₘₐₓ for the crystalline formulation, which was dosed 6-96 times higher, was about 122 for the 1800 mg/day dose and about 178 for the 2400 mg/day dose. FIG. 15 summarizes the mean indibulin concentration profiles in breast cancer patients receiving the crystalline indibulin capsules under fasted conditions. FIG. 16 summarizes the mean indibulin concentration profiles in breast cancer patients receiving the SDD formulations under fasted conditions.

The ratio of Cₘₐₓ for the SDD tablets to the Cₘₐₓ of the crystalline capsules (1800 mg/day) is 16.9 and 17.6, for the 50 mg/day and 100 mg/day doses, respectively. The ratio of Cₘₐₓ for the SDD tablets to the Cₘₐₓ of the crystalline capsules (2400 mg/day) is 15.5 and 16.2, for the 50 mg/day and 100 mg/day doses, respectively. The ratio of AUC for the SDD tablets to the AUC of the crystalline capsules (1800 mg/day) is 13.5 and 15.3, for the 50 mg/day and 100 mg/day doses, respectively. The ratio of AUC for the SDD tablets to the AUC of the crystalline capsules (2400 mg/day) is 13.3 and 15.2, for the 50 mg/day and 100 mg/day doses, respectively. These data are consistent with the data observed in rats and confirm plasma exposures that are about 10-20 times higher with the SDD indibulin, compared to crystalline indibulin. These data, along with the rat data, suggest the oral bioavailability of SDD indibulin is at least about 10-20 times higher than that of crystalline indibulin. As with the data observed in the rat model, the in vivo data suggest unexpectedly enhanced bioavailability of the SDD formulations. Moreover, the Cₘₐₓ at 100 mg (QD) of the SDD tablet formulation is comparable to that at 18-24 times the dose of the crystalline capsule formulation. Specifically, the Cmax values in patients receiving 100 mg of the SDD formulation were comparable to the Cmax values for patients receiving a dose that was 18 times higher in the crystalline formulation, dosed three times per day. Additionally, exposures were less variable for the SDD formulation compared to the crystalline formulation.

The data for the 275 mg daily dose under fed conditions indicated that plasma Cmax and AUC were about 2-3× higher under fed conditions compared to the 275 mg daily dose under fasted conditions. Additionally, the Tₘₐₓ is delayed under fed conditions when compared to the Tₘₐₓ under fasted conditions. FIG. 17 summarizes the mean indibulin plasma concentrations following oral administration of SDD indibulin tablets in breast cancer patients under fed conditions. For comparition, also shown is the mean plasma concentration for the 275 mg dose administered under fasted conditions.

TABLE 20
Mean Indibulin Plasma Pharmacokinetics
in Patients (fed conditions).

| | |
|---|---|
| | Breast Cancer Patients (Mean ± SD) |
| Formulation | SDD Formulated Indibulin (Tablets) |

| | | |
|---|---|---|
| Dose (mg/day) | 275 (n = 2)* | 350 (n = 3) |
| Dosing Regimen | QD, fed | QD, fed |
| Tmax (hr)⁺ | 5.0 | 4.0 ± 2.00 |
| Cmax (ng/mL) | 294 | 249 ± 25 |
| AUC₀₋₂₄ | 3556 | 2904 ± 529 |
| (hr · ng/mL) | | |
| Cₘₐₓ (ng/mL)/ | 1.1 | 0.7 ± 0.1 |
| Dose (mg) | | |
| AUC₀₋₂₄ | 12.9 | 8.3 ± 1.5 |
| (hr · ng/mL)/ | | |
| Dose (mg) | | |

*One subject had abnormal concentration profile and was excluded from the group mean.

### EQUIVALENTS

All of the above-cited references and publications are hereby incorporated by references in their entirety. Particular references incorporated herein by reference in their entirety include U.S. Pat. Nos. 6,232,327 and 6,693,119, and U.S. Patent Application Publication Nos. 2006/0280787, 2008/0241274 and 2006/0110462 and PCT International Publication No. 2011/028743.

Those skilled in the art would readily appreciate that all parameters and examples described herein are meant to be exemplary and that actual parameters and examples will depend upon the specific application for which the composition and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. Accordingly, those skilled in the art would recognize that the use of a composition or method in the examples should not be limited as such. The present invention is directed to each individual composition, or method described herein. In addition, any combination of two or more such compositions or methods, if such composition or methods are not mutually inconsistent, is included within the scope of the present invention.

1. A spray-dried solid dispersion comprising indibulin and at least one matrix polymer.

2. A spray-dried solid dispersion comprising indibulin and at least one matrix polymer.

3. The spray-dried solid dispersion of claim 1, wherein the at least one matrix polymer is hydroxypropyl methylcellulose acetate succinate (HPMCAS).

4. The spray-dried solid dispersion of claim 1, wherein indibulin is present in an amount in the range of about 5% to about 60% by weight.

5. (canceled)

6. (canceled)

7. (canceled)

8. The spray-dried solid dispersion of claim 1, wherein the dispersion is an amorphous powder.

9. The spray-dried solid dispersion of claim 1, comprising whole and collapsed spherical particles.

10. The spray-dried solid dispersion of claim 1, wherein the solid dispersion is a plurality of particles having an average diameter of less than about 100 microns.

11. The spray-dried solid dispersion of claim 1, wherein the dispersion has at least one of the following properties: a bulk specific volume in the range of about 2.5 to about 8.0 cm³/g, a tapped specific volume in the range of about 1.5 to about 4.5 cm³/g, and a glass transition temperature (T_{g}) of the dispersion ranges from about 50 to about 125° C.

12. (canceled)

13. (canceled)

14. The spray-dried solid dispersion of claim 1, wherein orally administering the dispersion to a rat or a human results in a maximum plasma drug concentration (plasma Cₘₐₓ) of indibulin that is higher by a factor of at least 5 relative to the plasma Cₘₐₓ of a control composition comprising an equivalent quantity of bulk crystalline indibulin following oral administration or in an area under the curve (AUC) for plasma indibulin that is higher by a factor of at least 5 relative to the AUC of a control composition comprising an equivalent quantity of bulk crystalline indibulin following oral administration.

15. (canceled)

16. The spray-dried solid dispersion of claim 13, wherein the dispersion plasma Cₘₐₓ is at least 10-fold higher relative to the control composition plasma Cₘₐₓ.

17. (canceled)

18. A process for making a spray-dried solid dispersion according to claim 1, the process comprising the steps of
(a) forming a solution comprising indibulin, at least one matrix polymer, water and a water-miscible solvent in which both indibulin and the at least one matrix polymer are soluble; and
(b) spray-drying the solution of step (a).

19. A spray-dried solid dispersion produced by the process of claim 17.

20. The process of claim 17, wherein the solvent is tetrahydrofuran (THF).

21. The process of claim 17, wherein the THF and water used in step (a) are in a ratio in the range of 90:10 to 99:1 w/w.

22. (canceled)

23. An oral dosage formulation comprising the spray-dried solid dispersion of claim 1.

24. The oral dosage formulation of claim 22, wherein the formulation is a tablet.

25. The oral dosage formulation of claim 22, comprising a filler, a disintegrant, a glidant and a lubricant.

26. The oral dosage formulation of claim 22, wherein the spray-dried solid dispersion is present in an amount from about 20 to about 80% by weight.

27. (canceled)

28. (canceled)

29. The oral dosage formulation of claim 22, wherein indibulin is present in an amount from about 10 to about 150 mg per dose unit.

30. (canceled)

31. (canceled)

32. (canceled)

33. A method for treating cancer, comprising administering to a subject in need thereof a spray-dried solid dispersion according to claim 1.

34. The method of claim 32, wherein the cancer comprises a drug-resistant or metastasizing carcinoma.

35. The method of claim 32, wherein the cancer is adenoid cystic carcinoma, renal cell carcinoma, breast cancer, ovarian cancer, prostate cancer, vulvar cancer, glioblastoma, or lung cancer.

36. (canceled)

37. The method of claim 32, further comprising conjointly administering to the subject one or more other therapeutic agents, wherein the combination shows efficacy that is greater than the efficacy of either agent administered alone.

38. The method of claim 32, wherein the other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone.

39. A method for inhibiting angiogenesis, comprising administering to a subject in need thereof a spray-dried solid dispersion according to claim 1.

40. The method of claim 32 wherein the spray-dried solid dispersion or oral dosage formulation is administered to a subject at a total daily dose of about 25 mg to about 450 mg.

41. (canceled)

42. A kit comprising the spray-dried solid dispersion according to claim 1, and a second formulation comprising at least one therapeutic agent selected from erlotinib, carboplatin, 5-fluorouracil, capecitabine, paclitaxel, tamoxifen, vinorelbine, cisplatin, gemcitabine, estramustine, doxorubicin, vinblastine, etoposide, prednisolone, palifosfamide, ifosfamide, or darinasparin.
